(19) United States
(12) Patent Application Publication
(10) Pub. No.: US 2022/0315878 A1
(43) Pub. Date: Oct. 6, 2022

(54) HIGH DENSITY BOTTLE DRUM FOR STORAGE, AGITATION AND READING OF BLOOD CULTURE BOTTLES AND METHODS OF STORING

(71) Applicants: Daniel Justin LOHAN, Glen Rock, PA (US); Kevin WENGER, Baltimore, MD (US); BECTON, DICKINSON AND COMPANY, Franklin Lakes, NJ (US)

(72) Inventors: Robert Edward Armstrong, Hunt Valley, MD (US); Brent Ronald Pohl, Timonium, MD (US); Ammon David Lentz, York, PA (US); Jason Zerweck, Media, PA (US); Christopher Murray, Philadelphia, PA (US); Daniel Justin Lohan, Glen Rock, PA (US); Kevin Wenger, Baltimore, MD (US)

(73) Assignee: BECTON, DICKINSON AND COMPANY, Franklin Lakes, NJ (US)

(21) Appl. No.: 17/632,912

(22) PCT Filed: Aug. 5, 2020
(86) PCT No.: PCT/US2020/045065

§ 371 Date: Feb. 4, 2022

Related U.S. Application Data
(60) Provisional application No. 62/883,796, filed on Aug. 7, 2019.

(51) Int. Cl. C12M 3/00; C12M 1/00; C12M 3/04; C12M 1/34

(52) U.S. Cl. CPC C12M 23/46; C12M 23/26; C12M 23/34; C12M 27/10; C12M 41/46

(57) ABSTRACT

An apparatus for storing and monitoring blood culture bottles. The apparatus has a moveable rack configured as a drum having a plurality of receptacles therein for receiving blood culture bottles. The drum is disposed in a housing. The housing includes a heater and a blower for incubating the blood culture bottles at elevated temperatures. Optionally the apparatus has a plurality of drums, each having a plurality of receptacles for receiving blood culture bottles.

### CROSS REFERENCE TO RELATED APPLICATIONS

The present application is a national phase entry under 35 U.S.C. § 371 of International Application No. PCT/US2020/045065, filed Aug. 5, 2020, published in English, which application claims the benefit of priority from U.S. Provisional Application No. 62/883,796 filed Aug. 7, 2019, which is incorporated by reference herein.

### TECHNICAL FIELD

The present invention relates to a non-invasive apparatus for detecting biological activities in a specimen such as blood, where a number of specimens with culture medium are introduced into a large number of sealable containers and are exposed to conditions enabling a variety of metabolic, physical, and chemical changes to take place in the presence of microorganisms in the sample. These changes are then monitored using colorimetric or fluorescent chemical sensors disposed to the inner bottom of each blood culture bottle as the bottles are rotated in a rotatable drum. After monitoring is complete, the apparatus performs “auto-unloading” and sorting of final negative and final positive bottles.

### BACKGROUND

The presence of biologically active agents such as bacteria in a patient's body fluid, especially blood, is generally determined using blood culture bottles. A small quantity of blood is injected through an enclosing rubber septum into a sterile bottle containing a culture medium, and the bottle is then incubated at about 35° C. and monitored for microorganism growth.

Since it is of utmost importance to learn if a patient has a bacterial infection, hospitals and laboratories have automated apparatus that can process many blood culture bottles simultaneously. One example of such an apparatus is the BD BACTEC™ system, which is manufactured and sold by Becton, Dickinson and Co. U.S. Pat. No. 5,817,508 to Berndt et al. describes a prior art blood culture apparatus, and is incorporated by reference herein. Additional descriptions of Blood Culture Apparatus are provided in U.S. Pat. No. 5,516,692 (“Compact Blood Culture Apparatus”) and U.S. Pat. No. 5,498,543 (“Sub-Compact Blood Culture Apparatus”) both of which are incorporated by reference herein.

Referring to FIG. 1, a culture medium and blood specimen mixture 22 are introduced into sealable glass bottles 1 that include optical chemical sensing means 20 on their inner bottom surface 21. Optical chemical sensing means 20 emanates differing quantities of light depending upon the amount of a gas in bottle 1. For example, the gas being detected by optical sensing means 20 can be carbon dioxide, oxygen or any gas that increases or decreases depending upon the presence or absence of microorganism growth in bottle 1.

As illustrated in FIGS. 1 and 2, a plurality of such bottles 1 are arranged radially on a rotating bell-shaped drum 2 within an incubator 5 in such a way that the bottoms of bottles 1 are oriented towards a drum axis 28. Bell-shaped drum 2 is hollow and is supported by a shaft 24 rotatably supported on one end by two large ball-bearings 3 and 4 mounted to a first side 51 of an instrument mainframe 50. In order to read information coming from each optical chemical sensing means 20 within bottles 1, a linear array of sensor stations 12 is mounted within rotating bell-shaped drum 2 to a second side 52 of instrument mainframe 50 at such a distance inside bell-shaped drum 2 that, during rotation of drum 2, individual bottles 1 are passing by respective sensor stations 15 in array 12. Each sensor station 15 of the linear array of sensor stations 12 comprises an excitation light source 11 and a collection end of an optical fiber 14.

Axis 28 of the bell-shaped drum 2 is oriented horizontally and parallel to a door 13, shown in FIG. 2, located on a front face of incubator 5. Horizontal orientation of axis 28 provides maximum agitation of the liquid culture medium and specimen mixture 22 and the gas within each bottle 1. During a load or unload operation, door 13 is opened which allows to access approximately one third of all bottles 1 simultaneously. Then, drum 2 is rotated until the next third of bottles 1 becomes accessible. In three steps, all bottles 1 are accessible.

Alternatively, axis 28 of bell-shaped drum 2 is oriented vertically with a slight tilting of approximately 20 degrees away from door 13. By adjusting the tilt angle, the degree of agitation can be modified, if required, for maintaining optimum growth conditions.

In operation, bell-shaped drum 2 is rotated by motor 6 and a belt 7. A circular member 8 and a sensor 9 form an angular encoder that provides information about which row of bottles 1 is passing sensor station array 12. Preferably, motor 6 is a stepper motor, allowing drum 2 to rotate either in a continuous mode or to stop drum 2 at appropriate angles to read from sensing means 20 within bottles 1 in a steady-state mode. The whole system is controlled by a control system 10 located inside rotating drum 2. Output ends of all optical fibers 14 of the linear array of sensor stations 12 are fed to one common photodetector (not shown) in control system 10 such that only one excitation light source 11 needs to be turned on at a time. Therefore, the control system “knows” from which sensing station 15 and, therefore, which bottle 1 the sensor light is being collected.

The apparatus shown in FIGS. 1 and 2 contains ten segments of blood culture bottles 1 with thirty-six bottles 1 per segment. Consequently, the total capacity is 360 bottles. The arrangement of bottles 1 on drum 2 allows for a relatively high packaging density, but improvement in density is still sought.

### BRIEF SUMMARY

Described herein is an apparatus for storing and monitoring blood culture bottles. The apparatus has a moveable rack configured as a drum having a plurality of receptacles therein for receiving blood culture bottles. The drum is disposed in a housing. The housing includes a heater and a blower for incubating the blood culture bottles at elevated temperatures. Optionally the apparatus has a plurality of drums, each having a plurality of receptacles for receiving blood culture bottles.

Blood culture bottles typically have a bottom portion that is the larger volume of the bottle and a neck portion which is the narrower top portion of the bottle. The drum receptacles can be configured to receive the blood culture bottles either bottom-inward (bottom-in) or neck-inward (neck-in). In the bottom-in configuration the receptacles are typically angled downward so that the culture bottles are held at an upward angle of 20° from horizontal. However, the culture bottles can be held horizontally (i.e. the neck portion is not angled upward or downward relative to the bottom portion) and accurate measurements of the blood culture bottle contents to determine whether the contents are positive for microbial growth can be obtained. To obtain a high density of bottles, the drums are configured to rotate around an axis with the circular drum rack situated co-axially with the axis of rotation. Electronics for monitoring the blood culture bottles are disposed in the drum interior, if the culture bottles are placed in the drum bottom side in. If the bottles are placed in the drum neck end first, then some measurement electronics are placed on the outside of the drum.

The drums are formed in a modular manner. The drums have multiple rows of receptacles and multiple columns of receptacles. In one configuration, a row of receptacles is formed by obtaining molded articles that are upper and lower portions of a row. The molded articles are assembled to form one row of drums and multiple rows are assembled together to form the drum. Optionally the receptacles are aligned vertically in the assembled drum. Optionally, the receptacles are staggered in the assembled drum which provides a greater bottle density in the drum.

The drum has both an external perimeter and an internal perimeter. The receptacles receive the culture bottles therein and the receptacles extend from the first perimeter to the second perimeter. The receptacles are evenly distributed in rows that travel along the perimeter of the drum.

In those embodiments where the drum receptacles receive the bottle neck in, the receptacle includes a mechanism to retain the bottle in the receptacle. This mechanism can be a spring clamp, a cap clamp, a flat spring or a stop. Such mechanisms allow the culture bottles to be securely held in the drum during drum operation.

Described herein is an apparatus for storing and monitoring blood culture bottles. The apparatus has a housing with a drum therein. The drum has an exterior perimeter and an interior perimeter. The exterior perimeter has a diameter in excess of a diameter of the interior perimeter. The drum has a plurality of receptacles, the receptacles having a proximal end at the exterior perimeter and a distal end at the interior perimeter, each receptacle configured to receive a blood culture bottle. The blood culture bottle has a bottom portion and a neck portion, wherein the bottle can be received by the receptacle such that either the bottom portion is received at the distal end of the receptacle or the neck portion is received at the distal end of the receptacle.

The drum perimeters are disposed about an axis of rotation of the drum. The plurality of receptacles is disposed in the drum as an array of receptacles, the array having receptacles disposed both vertically and horizontally. The apparatus also has sensors and detectors for interrogating the blood culture bottles to determine if the blood culture bottles are positive or negative for microbial growth. The drum defines an interior space within the interior perimeter wherein at least a portion of drum electronics in communication with the sensors and detectors for interrogating the blood culture bottles are disposed in the interior perimeter of the drum.

Optionally, the plurality of receptacles each have a respective elastomeric insert for receiving a culture bottle. Optionally, each of the plurality of receptacles each receive the neck portion of the culture bottle at the distal end and each receptacle comprises a bottle stop and a canted coil spring spaced apart from the bottle stop. The canted coil spring is biased to permit a cap disposed on the neck portion of the bottle to pass by the canted coil spring and proceed to rest against the bottle stop, whereby the canted coil spring releases tension and secures the culture bottle in the receptacle.

Optionally, the plurality of receptacles each receive the neck portion of the culture bottle at the distal end and each receptacle comprises a bottle stop and an o-ring spaced apart from the bottle stop. The o-ring is sufficiently elastic to permit a cap disposed on the neck portion of the bottle to pass by the o-ring and proceed to rest against the bottle stop. The o-ring then releases tension and secures the culture bottle in the receptacle.

Optionally, the plurality of receptacles each receive the neck portion of the culture bottle at the distal end and each receptacle has a bottle stop and a ball plunger spaced apart from the bottle stop. The ball plunger is biased to permit a cap disposed on the neck portion of the bottle to pass by the ball plunger and proceed to rest against the bottle stop. The ball plunger then releases tension and secures the culture bottle in the receptacle.

Optionally, the plurality of receptacles each receive the neck portion of the culture bottle at the distal end and each receptacle comprises a bottle stop and a segmented retention portion spaced apart from the bottle stop. The segmented retention portion is biased to permit a cap disposed on the neck portion of the bottle to pass into the segmented retention portion and proceed to rest against the bottle stop. The segmented retention portion releases tension and secures the culture bottle in the receptacle. Optionally, the segmented retention portions are urged together by a canted coil spring or the segmented retention portions are resilient segments.

Optionally, the plurality of receptacles each receive the neck portion at the distal end and each receptacle has a bottle stop and a plurality of resilient wings extending from the bottle stop. The wings are biased to permit a cap disposed on the neck portion of the bottle to pass past a flange portion of the wings and proceed to rest against the bottle stop. The wings release tension and the flange portion secures the culture bottle in the receptacle. Optionally, each of receptacles with resilient wings have a ball plunger. The bottle stop has a notch. As the culture bottle advances into the bottle stop, the bottle stop is advanced further into the receptacle until the ball plunger aligns with the notch, thereby securing the bottle stop in the receptacle.

Optionally, the plurality of receptacles has a notch and the bottle stop has a ball plunger. As the culture bottle advances into the bottle stop, the bottle stop is advanced further into the receptacle until the ball plunger aligns with the notch, which secures the bottle stop in the receptacle.

Optionally, the plurality of receptacles has a tray portion on which the culture bottle is placed. The tray portion has a tab for securing the bottom of the culture bottle in the tray. The receptacle also has a stop portion. The stop portion places the culture bottle in a predetermined fixed position within the receptacle. The stop portion can be one of a leaf spring, a deformable material and/or a pivoting arm that pivots in response to the culture bottle being advanced into the receptacle, thereby securing the culture bottle in the receptacle. the stop portion may be made of a resilient material selected from one of elastomeric flexible tubing, elastomeric materials, or foam materials.

Optionally, the tray portion of the receptacle forms a light pipe that transmits a light signal from indicator LEDs disposed within the inner perimeter of the drum to be visible from the exterior of the drum. The bottle stop can also be a keyhole element. Optionally, the exterior perimeter of the drum and the interior perimeter of the drum are circular.

A method for controlling an incubator for a plurality of blood culture bottles is described herein. In the method, an operator inputs, via a control interface, a predetermined sector of the blood culture drum. The blood culture drum is a rack that defines a substantially circular interior perimeter and a substantially circular exterior perimeter. The blood culture drum is rotatable. A door of a housing for the blood culture drum is opened by the operator and the drum is stopped, typically by reducing power to a motor used to rotate the drum such that the predetermined sector is accessible via the opened door of the housing. The blood culture drum has a plurality of receptacles, each receptacle of the plurality of receptacles being adapted to receive and retain a blood culture bottle. Each receptacle of the plurality of receptacles has an indicator at a proximal end thereof. The indicator provides an indication that a culture bottle in the receptacle is positive or negative for microbial growth. The blood culture bottles are either inserted into an empty receptacle of the plurality of receptacles, removed from a receptacle of the plurality of receptacles, or removed from one receptacle of the plurality of receptacles after which another blood culture bottle is inserted in its place. The blood culture bottles are removed based on their indicated status.

Optionally, the receptacles each have a light pipe extending from the proximal end of the receptacle to a distal end of the receptacle. Optionally, the control interface is in communication with an encoder such that the control interface tracks a placement of each blood culture bottle in the rack. Optionally, the incubator further comprises a reader station, wherein the reader station determines the status of the blood culture bottle as the bottle drum rotates the receptacles carrying blood culture bottles past the reader station. Optionally, indicator LEDs are positioned at the distal end of at least a portion of the receptacle in the bottle drum. As noted elsewhere herein, the culture bottles are received in each of the plurality of receptacles either neck-in or bottom-in. As such the receptacles are either configured to receive the culture bottles neck-in or bottom-in.

### BRIEF DESCRIPTION OF THE DRAWINGS

FIG. 1 shows a front-view of the interior of a blood culture apparatus for the detection of microorganisms of the prior art;

FIG. 2 shows a side-view of the interior of a blood culture apparatus of the prior art;

FIGS. 3A and 3B illustrate perspective views of a blood culture apparatus housing for the module described herein;

FIG. 4 is a top view of an incubation and measurement module according to one embodiment of the present invention;

FIGS. 5A-5B are cross-sections of an incubation and measurement module drum;

FIGS. 6A-B illustrate an example of an actuator for the incubation and measurement module;

FIG. 7 is a schematic of culture bottles placed bottom-in fit in a high-density drum described herein;

FIG. 8 is a cross section of one embodiment of a drum described herein with the culture bottles placed bottom-in;

FIG. 9 illustrates three stacked drums;

FIG. 10 illustrates a different drum design with the culture bottle facing neck-in;

FIG. 11 is a cross section of the neck-in embodiment of the drum illustrated in FIG. 10;

FIG. 12 is a stack of the drums described in FIG. 11;

FIG. 13 illustrates a bottle drum of the present invention and an exploded view of one tier of the bottle drum;

FIG. 14 illustrates another embodiment of a bottle drum of the present invention and an exploded view of one tier of the bottle drum configuration;

FIG. 15A-B are “neck-in” drum configuration assembled using vertical sections;

FIG. 16A-B is an alternate embodiment of a “neck-in” drum configuration assembled from vertical sections;

FIG. 17A-G illustrate bottle retention mechanisms for a “neck in” drum configuration;

FIG. 18A-C illustrate a bottle retention mechanism according to an alternative embodiment;

FIG. 19A-Q illustrate bottle retention mechanisms according to a different embodiment;

FIG. 20A-H is an alternate bottle retention mechanism integrated with a light pipe;

FIG. 21 is a detail view of FIG. 20A-C;

FIG. 22 illustrates an unseated bottle in the drum;

FIG. 23 illustrates an alternate drum configuration;

FIG. 24 illustrates a BCP response curve obtained from an embodiment of the apparatus described herein;

FIG. 25 illustrates one example of a ring bearing;

FIG. 26 illustrates another bottle retention mechanism;

FIG. 27 illustrates a culture bottle cap that carries information about the status of the culture bottle for use by an operator or user;

FIG. 28A-B illustrate a drum formed of the receptacles illustrated in FIGS. 20A-C; and

FIG. 29 illustrate one row of a drum formed of the receptacles illustrated in FIGS. 17A-C.

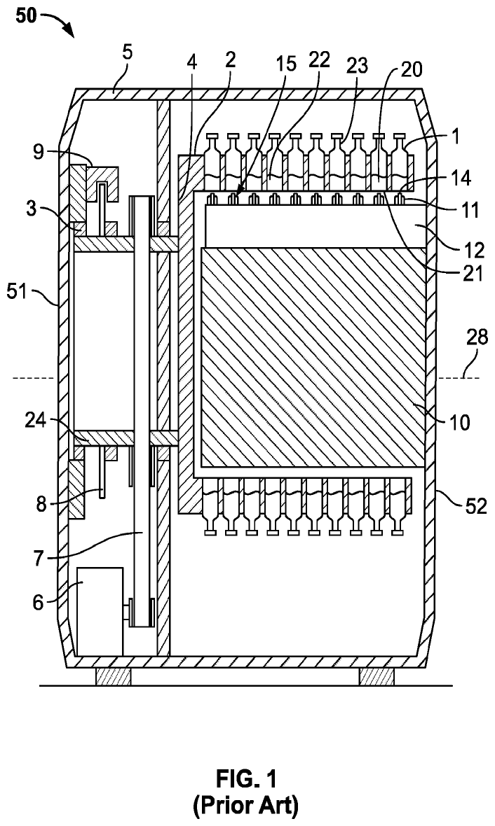

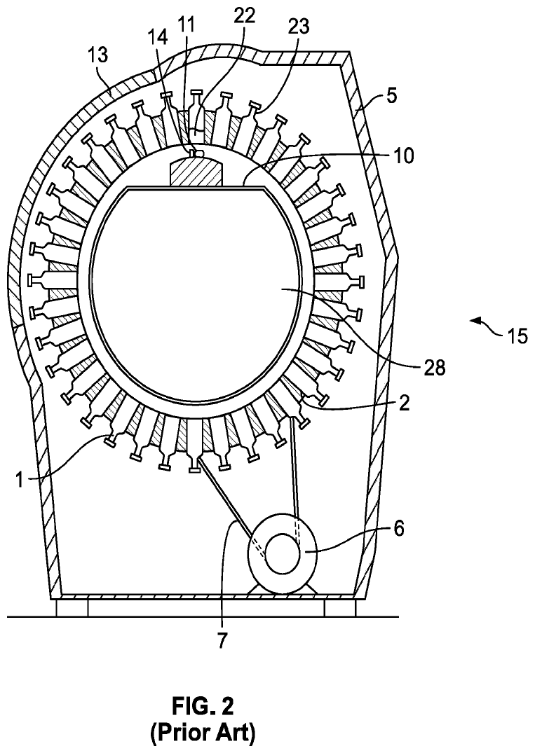

### DETAILED DESCRIPTION

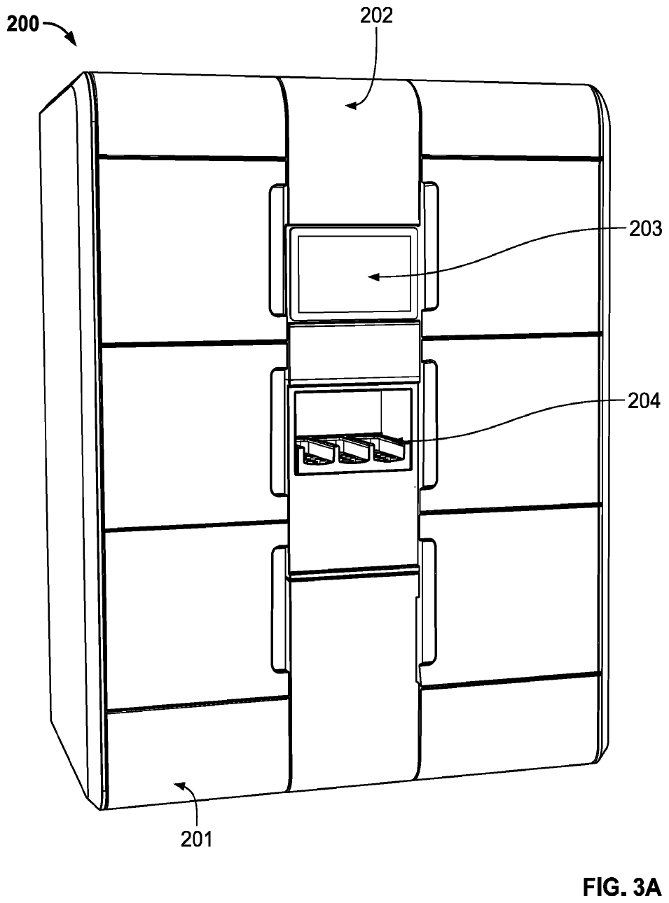

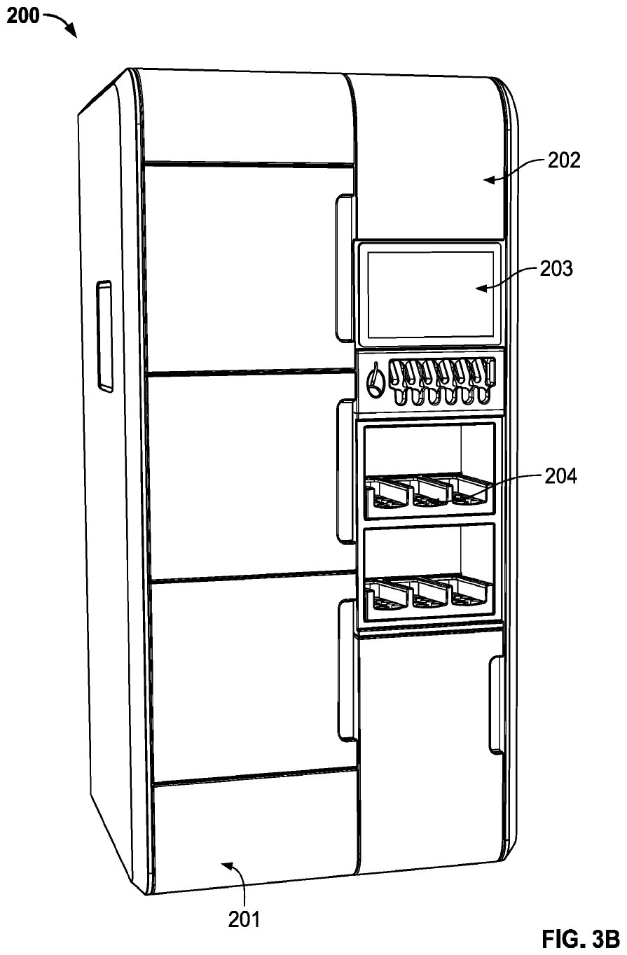

Described herein is a blood culture apparatus configured as an incubation and measurement module that, optionally, can be integrated with a larger end to end solution for processing biological samples to determine if such samples contain are contaminated with or infected by microorganisms. The module described herein can be placed in a cabinet such as is illustrated in FIGS. 3A and 3B. The cabinet 200 can provide power to the module, provide a controlled thermal environment to the module and a communication channel to the module. FIG. 3A illustrates a cabinet 200 with two three-door panels 201 that provide access to three bottle drums on each side of a central panel 202. Central panel 202 has touch screen 203 for data entry and user control. Central panel 202 also has a central station 204 for culture bottle input/output. FIG. 3B illustrates a cabinet with only one three-door panel 201.

The module has a high-density bottle drum. High density, as used herein is a description of drum configurations that allow culture bottles to be placed closer to each other to allow a greater number of bottles to be fitted into the drum compared to the prior art. The module is configured to align bottles with a limited number of reader stations. That is, the number of reader stations is less than the number of bottle receptacles in the drum. Optionally the drum is operated by a direct drive motor that can cause accelerated and decelerated drum movement (i.e. a rocking movement, intermittent rotation, etc.). A heater and blower are provided in the drum housing. The heater and blower circulate warm air around the drum. Optionally, the heater and blower will be configured to keep the temperature of the contents of all culture bottles in the drum within a predetermined narrow range of a specific target temperature. The predetermined narrow range is ±1.5° C. of the target temperature. The specific target temperature is in the range of 30° C. to 40°. Optionally, the target temperature is 35° C. Greater temperature uniformity will permit an increase in set point as there is less risk of “over-heating” samples. A greater temperature uniformity at higher temperature will therefore permit a faster time to detection. The motor will permit the drum to be positioned such that the user or the automated apparatus can access any bottle carried by the drum. When the sample in a bottle is determined to be positive for microbial growth, a workflow is activated to retrieve that culture bottle from the module. The module is configured to assist with that workflow.

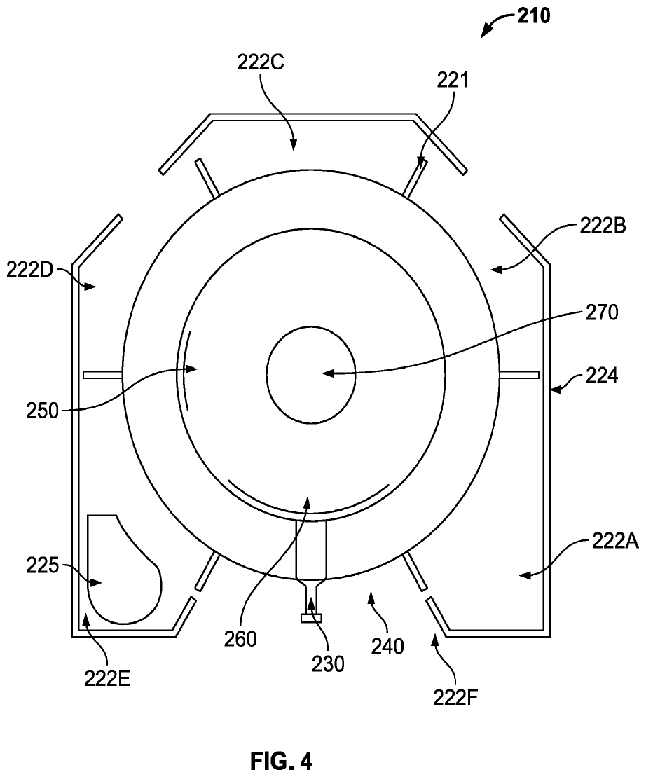

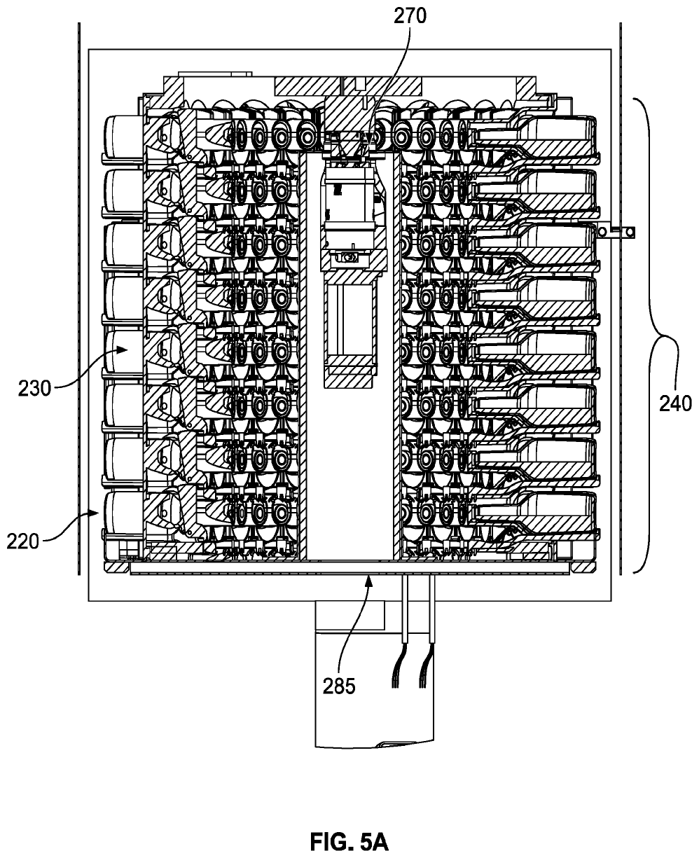

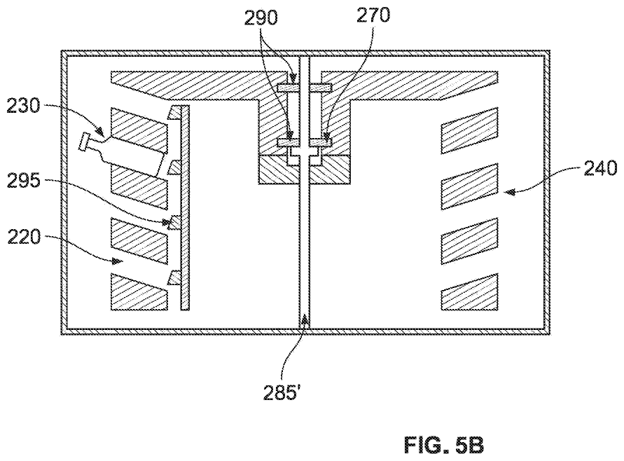

The module is configured to have LEDs and light pipes to indicate positive culture bottles to the user. Referring to FIG. 4, there is illustrated a top down view of an optional configuration of the module described herein. The module 210 has a housing 224, a blower and heater 225 for keeping the bottles 230 warm and a drum 240 with receptacles that hold the culture bottles. Positioned in the interior of the drum are measurement electronics 250 and culture bottle detection/indicator electronics 260. A drive motor 270 is provided to rotate the drum 240. The drum 240 housing 224 has six panels 221 that define six drum sectors (222A-222F). As illustrated, about one-sixth of the drum contents (assuming the drum is full) are available for access at any given time since the span of one sector is about the same as the span of the opening in the housing through which bottles are added to or removed from the drum 240. FIG. 5A is a cutaway side view of the drum in FIG. 4. The culture bottles 230 are disposed neck inward in receptacles 220 in the drum 240. The motor 270 is a direct drive motor provides high torque, little to no hysteresis, low noise, reliability and simplicity.

FIG. 5B is a cross-section of an incubation and measurement module drum with a stepper motor and gearbox. Alternatively, if correct sizing and spacing between of internal gearbox bearings cannot be achieved in the limited space constraints inside the drum an additional bearing set may be included towards the bottom of the drum ring.

As illustrated in FIG. 5A the motor drives the drum to rotate axially. Bearings inside the gearbox of the motor 270 provide both axial alignment of the drum 240 but also the requisite thrust load support required to advance the drum carrying a significant number of bottles 230. Alternatively, if correct sizing and spacing between of internal gearbox bearings cannot be achieved in the limited space provided in the interior of the drum perimeter an additional bearing set may be included towards the bottom of the drum ring. Mechanisms that can rotate the drum 240 about an axis are well known to one skilled in the art and are not described in detail herein. This structure and bearing configuration are adaptable to other drum configurations (e.g., a drum that receives culture bottles neck out instead of neck in; drums where the receptacles are angled upward, etc.). Optionally, the measurement electronics, the status indicator and bottle detection electronics are all supported by the single axle 285.

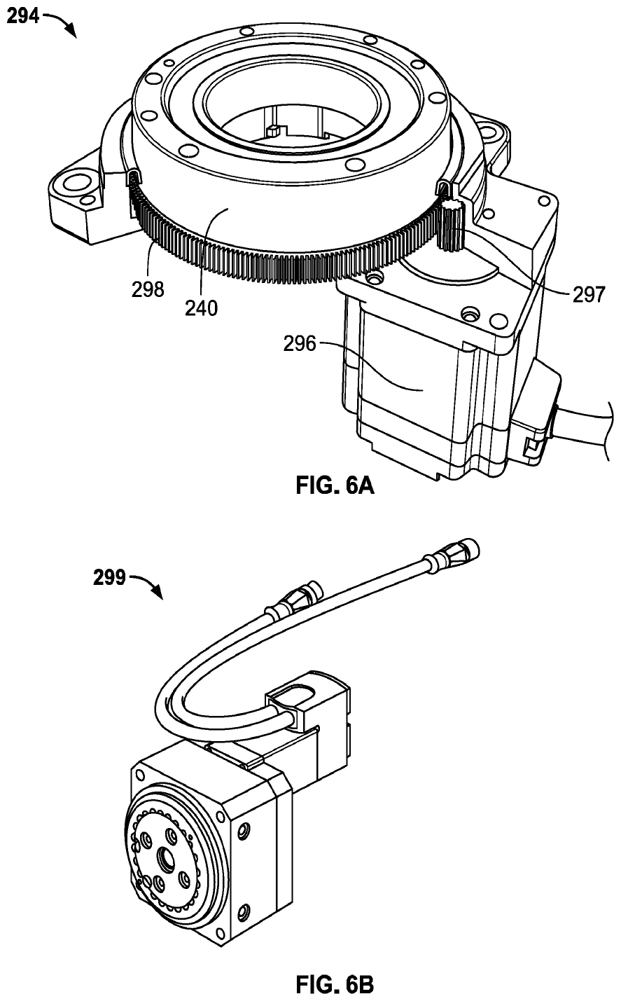

In FIG. 5A, a rotary actuator, hollow rotary actuator or stepper motor with gearbox is used to combine the bearing and motor functions into one component. Examples of such suitable motor actuators are illustrated in FIG. 6. FIG. 6A is a hollow rotary actuator 294 having a motor 296 that drives a pinion gear 297. The rotation of the pinion gear 297 causes the drum gear 298 to rotate the output table 239, thereby rotating the drum 240 (not shown). FIG. 6B is a rotary actuator 299 that directly causes the drum 240 (not shown) to rotate. Other examples of suitable motors with a gearbox are contemplated. Such motors can be directly attached to the gearbox (usually at the factory). Such a gearbox can reduce motor speed while increasing torque at the output shaft. The output shaft can be mechanically attached to a circular spacer block that has attachment points suitable for interfacing with drum attachment points.

FIG. 5B illustrates the culture bottle 230 positioned in the drum 240 at a 20° angle above horizontal. The skilled person is aware that a sensor (not shown) in the culture bottle must be fully covered with media during a reading. Other angles that meet these criteria are contemplated but the bottle angle must also accommodate a high-density design.

One embodiment could include the top bearing 290 in proximity to the top of the drum (240) and the second bearing 290 toward the bottom of the drum 240. Optionally, the first (top) bearing is a thrust ball bearing and the second (lower) bearing is a spherical bearing to accommodate any non-coaxial relationship between the first and second bearings. These structure and bearing configurations are adaptable to other drum configurations (e.g., a drum that receives culture bottles neck in instead of neck out). The motor 270 is a direct drive motor provides high torque, little to no hysteresis, low noise, reliability and simplicity. As illustrated in FIG. 5B a single axle 285′ supports the bottle drum 240 and drive motor 270. The two bearings 290 in between the axle 285 and the drum 240 are similar to the two bearings that support the rotor in prior art instruments. The distance between the two bearings can be increased to minimize the movement of the inner surface of the bottle drum relative to the axle. These structure and bearing configurations are adaptable to other drum configurations (e.g., a drum that receives culture bottles neck in instead of neck out). Optionally, the measurement electronics, the status indicator and bottle detection electronics are all supported by the single axle 285.

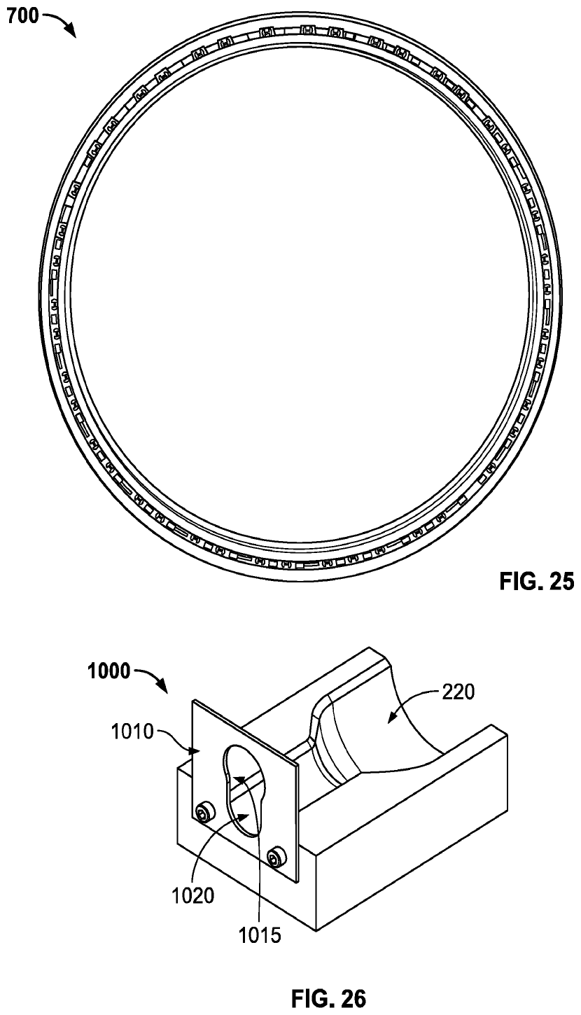

An alternative embodiment deploys a top-mounted ring bearing, which is smaller than ring bearings in prior art systems to guide the drum rotation. A ring bearing 700 is illustrated in FIG. 25. A bottom mounted ring bearing 600 is illustrated in FIG. 23. One of ordinary skill in the art could replace bearings 290 with a ring bearing that supports the drum from the top and is attached to the module housing 224.

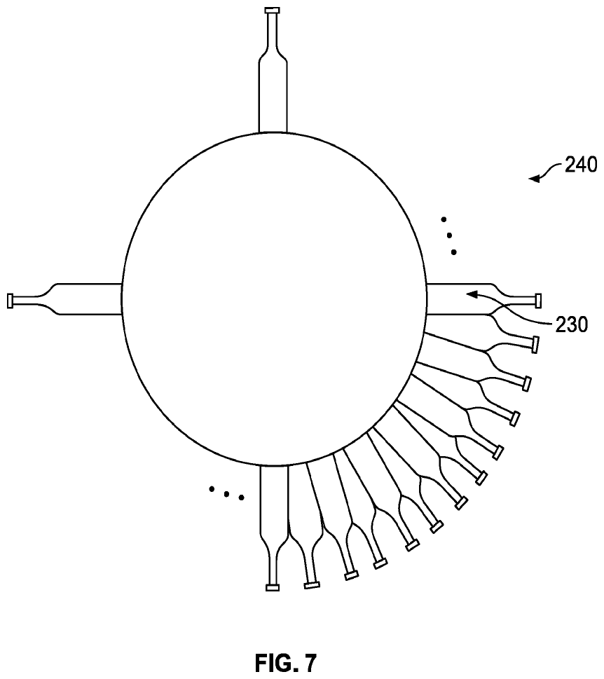

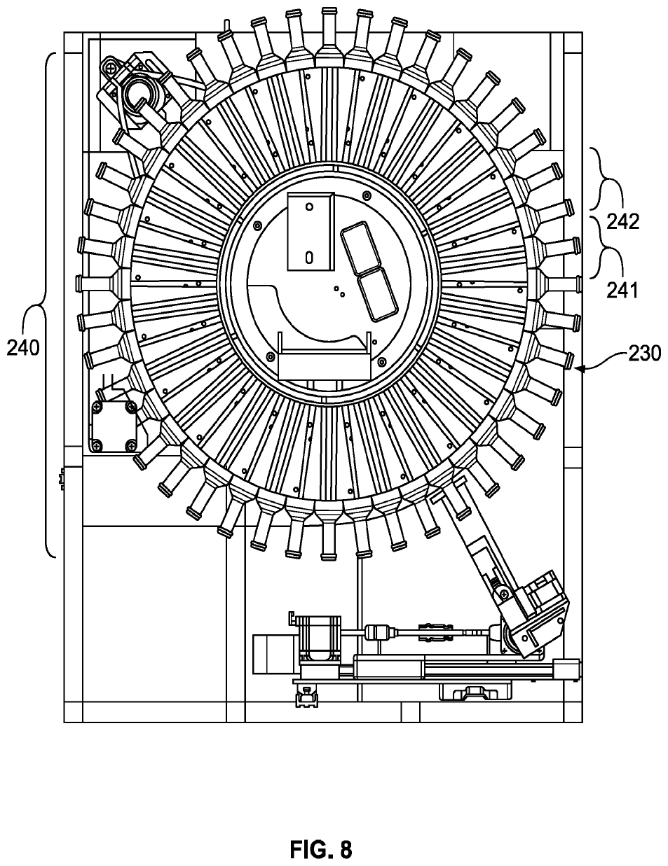

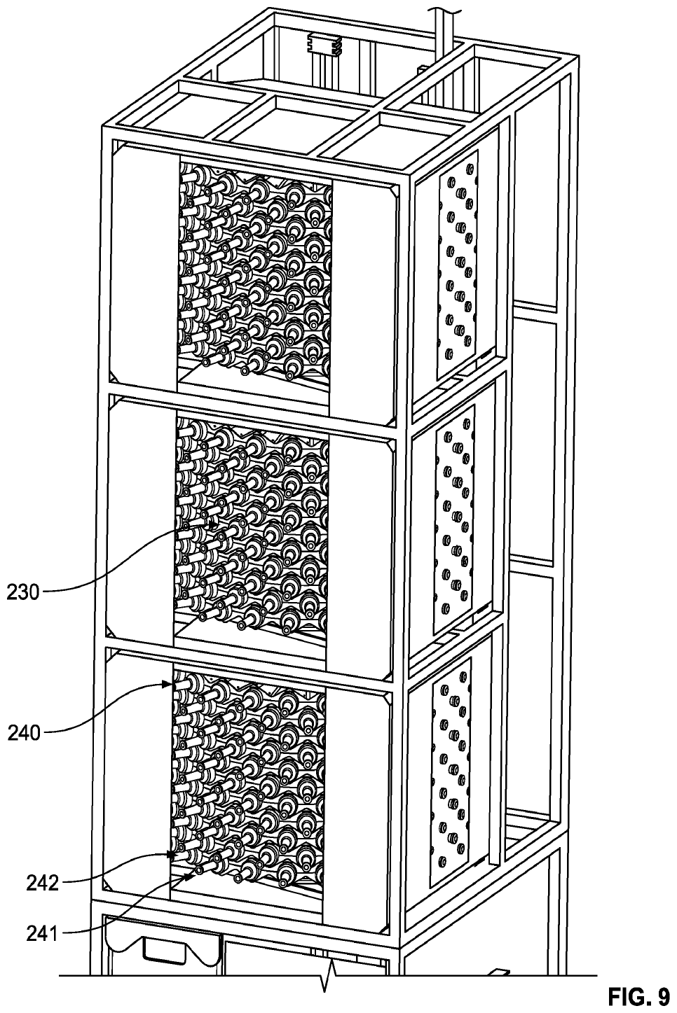

Referring to FIG. 7, the bottle drum 240 is a structure holding a tightly packed arrangement of culture bottles 230 with their necks facing out. The culture bottles 230 are arranged in staggered layers 241, 242 (FIG. 8) with their necks pointing out from the drum 240 to achieve the tight packing. FIG. 9 is a perspective view of the staggered drum illustrated in FIG. 8 in a frame configured for the three-door cabinet illustrated in FIGS. 3A and 3B. The staggered culture bottle placement also creates space above each culture bottle for labeling the culture bottle receptacles 220, and detecting the output of light-piped indicators 295 (FIG. 5B). Each bottle receptacle 220 will have a number indicating its horizontal position in the drum, and a status indicator to guide the user during manual workflow.

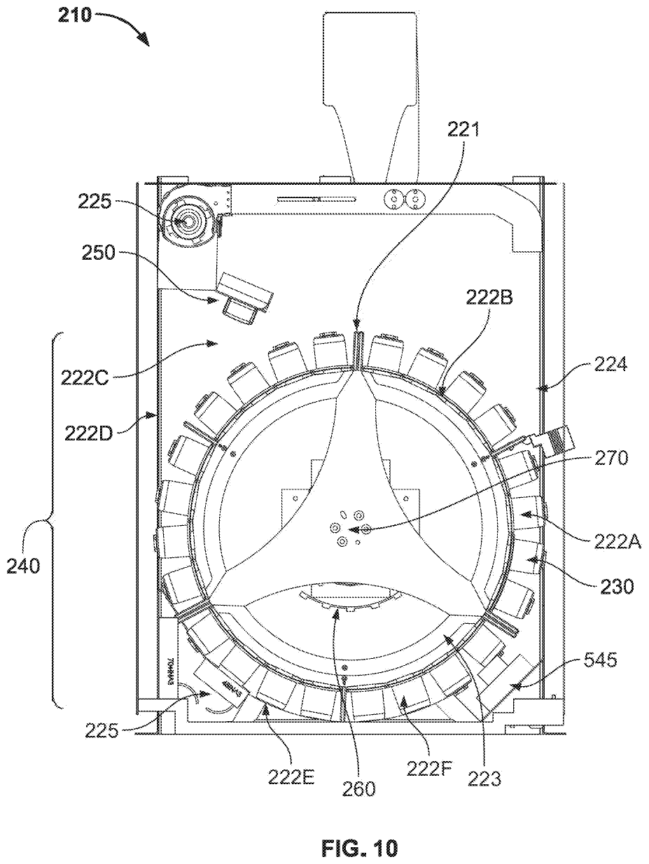

Referring to FIG. 10, the bottles 230 (facing inward) in the drum 240 are placed into one of six sectors (222A-222F) in the module 210. The sectors are demarcated by vertical panels 221 that extend outwardly from the drum 240. The span between adjacent panels is approximately equal to the span of a door into the housing 224 to completely shield the user from the inside of the module when a section of culture bottles is being accessed by the user. The module 210 also includes a blower and heater 225 for keeping the bottles 230 warm. Positioned in the interior of the drum are measurement electronics 525 and culture bottle detection electronics 260. A drive motor 270 is provided to rotate the drum 240.

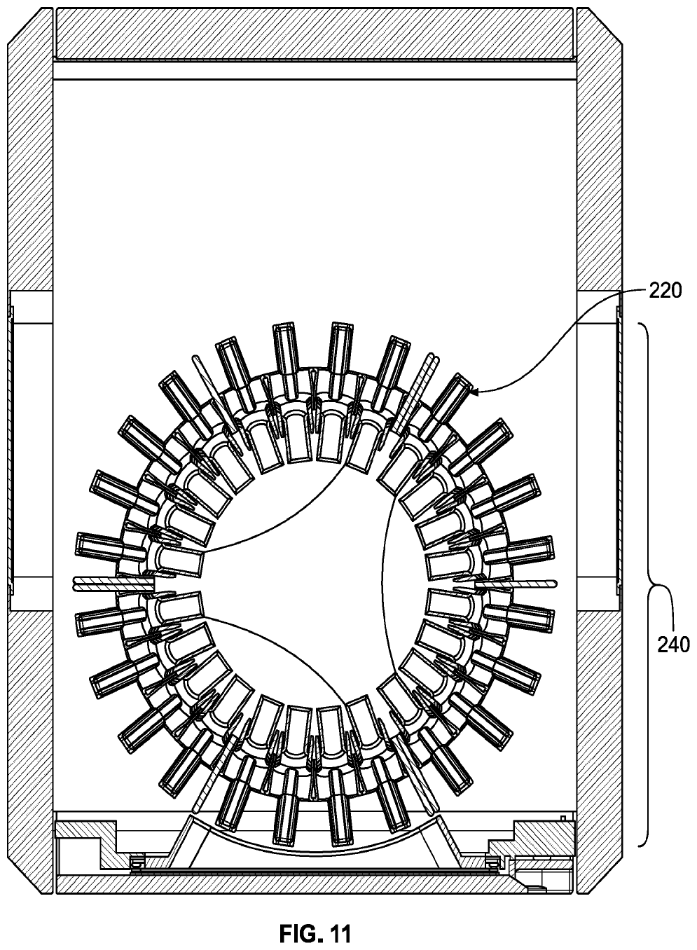

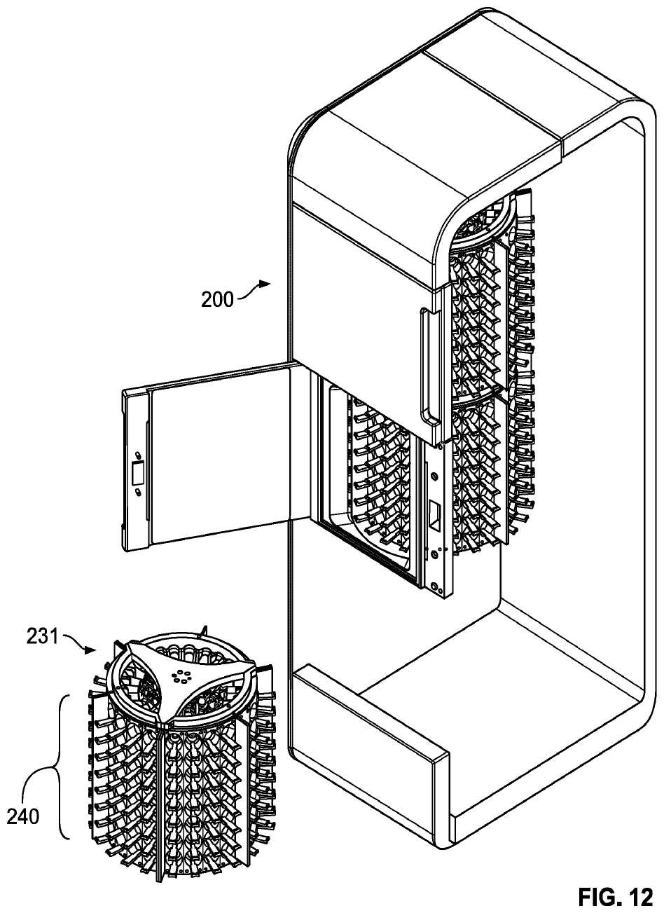

Referring to FIG. 11, the bottle drum 240 has receptacles 220 for receiving culture bottles disposed neck-in therein in layers as described above. The receptacles 220 have a light pipe formed in the bottom portion of the receptacle that also define the bottom edge of the receptacles. These features are described elsewhere herein. In FIG. 12, one bottle drum is removed from the three-door cabinet 200 described in FIG. 3. As shown in FIG. 11, there are twenty-four receptacles 220 that will receive twenty-four bottles 230 per layer 231, there are eight layers 231 per drum 240 and three drums 240, which is an about 576-bottle capacity. Optionally, the module has receptacles that will receive the bottles neck-out. In one embodiment, the drums will have thirty receptacles per layer, eight layers per drum and three drums, which is about 720-bottle capacity.

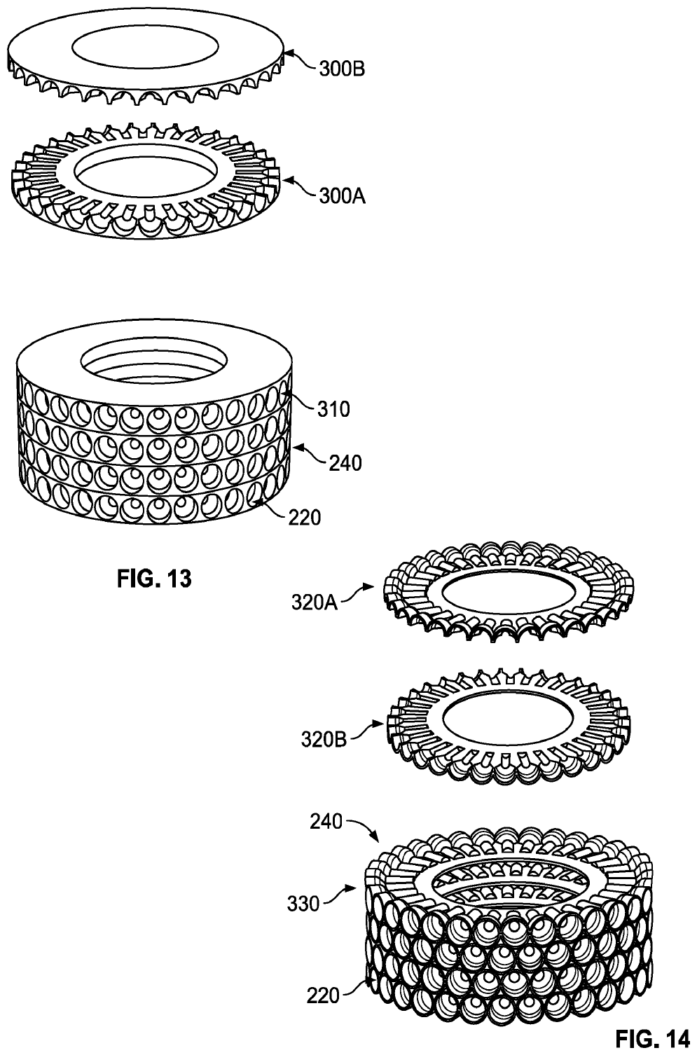

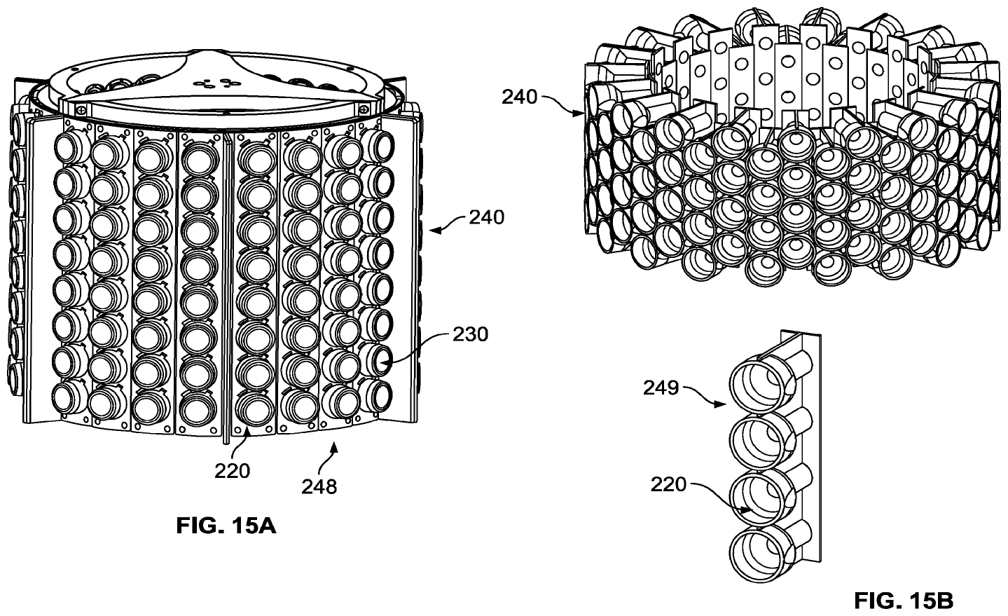

FIG. 13 illustrates a drum formed from injection molded layers 300A and 300B. When assembled together, the injected molded layers together form a single layer 310 of drum 240. Optionally the injection molded layers are molded to possess discrete retention features, cavities or other features that are provided to facilitate reception, retention and release of the culture bottles in the receptacles. Molding the receptacle with such features reduces the number of discrete parts and simplifies the manufacturing process. Instead of the round geometry illustrated in FIG. 13, the drum section may have a multi-faceted internal and external circumference for either manufacturing reasons or user aesthetic reasons. A multi-faceted drum is illustrated in FIG. 15A.

FIG. 14 illustrates a drum formed from injection molded layers 320A and 320B. When assembled together, the injected molded layers together form a single layer 330 of drum 240. In the embodiment of FIG. 14, the molded layers 320A and 320B, when brought together, form a honeycomb stack of receptacles 220, which provides for an approximately twenty percent higher bottle density in the drum 240. Optionally the injection molded layers are molded to possess discrete retention features, cavities or other features that are provided to facilitate reception, retention and release of the culture bottles in the receptacles. Molding the receptacle with such features reduces the number of discrete parts and simplifies the manufacturing process.

The drums can also be assembled in vertical sections. Referring to FIG. 15A, a bottle drum 240 (similar in appearance to the drum illustrated in FIG. 13) is assembled in vertical sections 248 of bottle receptacles. The vertical section 248 forms linear rows and columns of bottle receptacles 220 with bottles 230 inserted neck in. Referring to FIG. 15B, a drum 240 (similar in appearance to the drum illustrated in FIG. 14) is assembled from vertical sections 249. The vertical section 249 forms linear columns of bottle receptacles 220 but staggered rows that provide for a higher capacity drum 240. The vertical sections can be formed by injection molding. The vertical sections either interlock or attach to a central ring. Optionally the injection molded layers are molded to possess discrete retention features, cavities or other features that are provided to facilitate reception, retention and release of the culture bottles in the receptacles. Molding the receptacle with such features reduces the number of discrete parts and simplifies the manufacturing process.

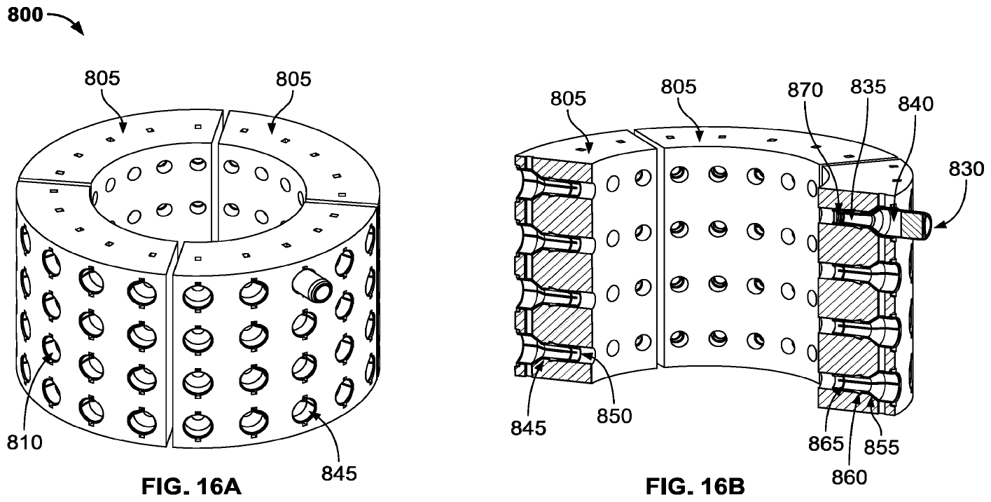

In the neck-in drum configurations, mechanisms in the culture bottle receptacles are required in order to retain and release the culture bottles. FIGS. 16A and 16 illustrate a drum 800 that is assembled in vertical segments 805 instead of horizontal segments as illustrated in FIG. 13. As noted above, the vertical segments 805 have at least one bottle receptacle 810 in each row. However, as illustrated in FIGS. 16A and 16B, the vertical segments 805 may have multiple bottle receptacles 810 in each row. The bottle receptacles 810 are configured to receive a bottle 830 with a narrow neck portion 835 and a wider bottle portion 840. Therefore, as illustrated, the bottle receptacles 810 have an outer portion 845 that has a diameter sufficient to receive the bottle portion 840 of the bottle and a narrower inner portion 850 with a diameter sufficient to receive the neck portion 835. As described elsewhere herein, there are many bottle retaining mechanisms contemplated herein. FIGS. 16A and 16B illustrate bottle receptacles 810 that have an elastomeric insert lining 855 in the bottle receptacles 810. The elastomeric insert lining 855 has a first flange 860 that retains the elastomeric insert 855 in the bottle receptacles 810. The elastomeric insert lining 855 has a second flange 865 positioned in the narrower inner portion 850 so that, when the bottle 830 is inserted into the receptacle 810, the second flange 865 will fit into a receptacle between a cap 870 placed on the bottle 830 and the neck portion 835 of the bottle 830. The second flange 865 retains the bottle 830 in the receptacle 810, but the force by which the second flange 865 holds the bottle 830 in the receptacle 810 can be easily overcome when the bottle 830 is to be removed from the bottle receptacle 810 of the drum 800.

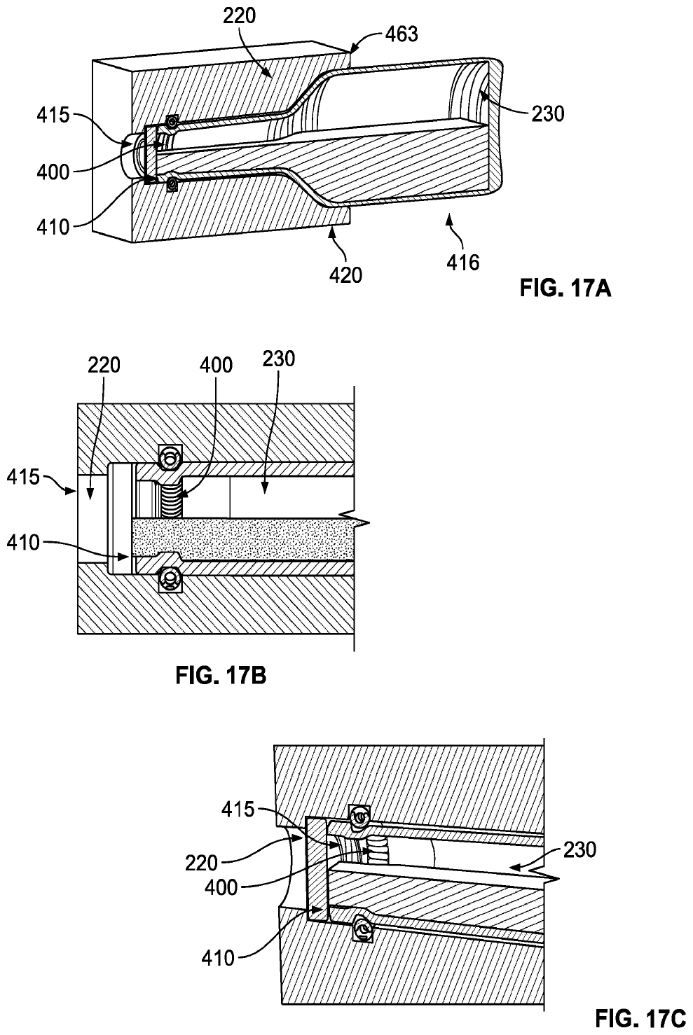

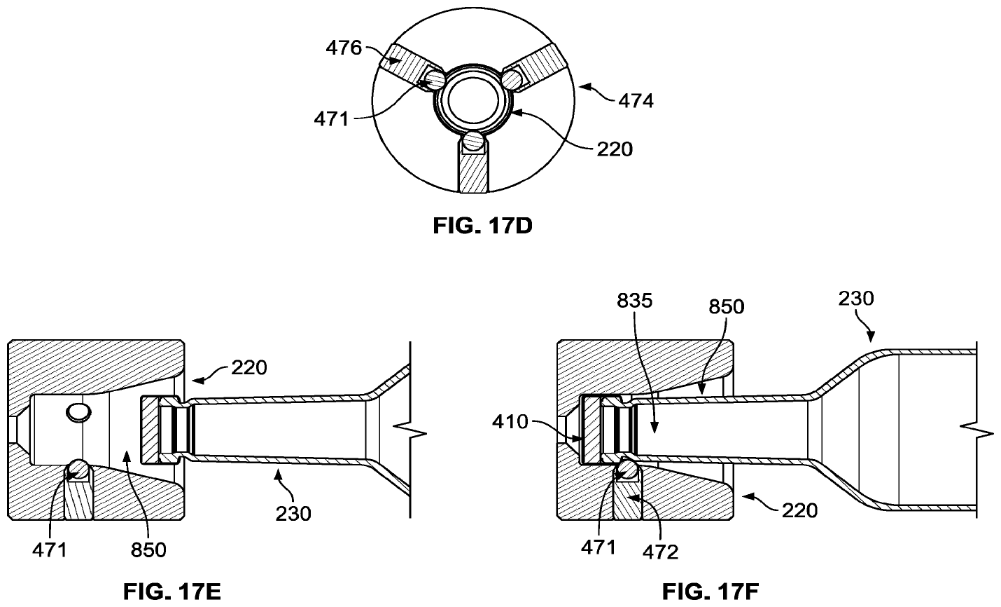

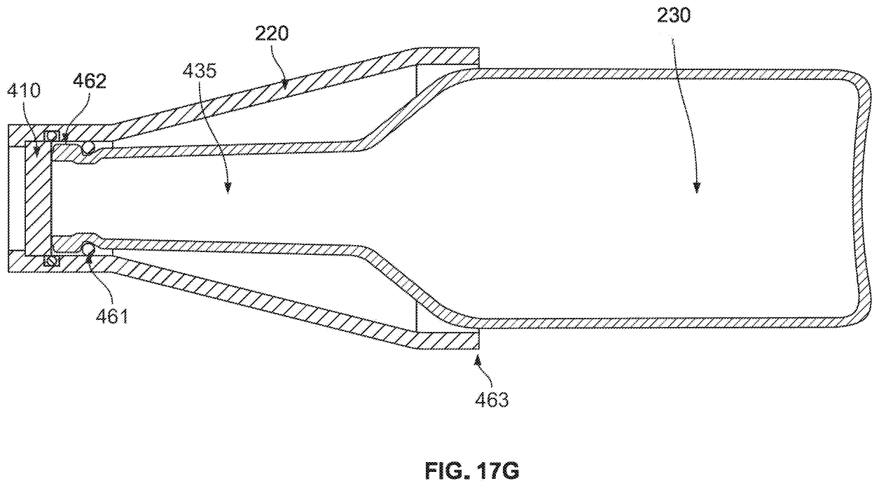

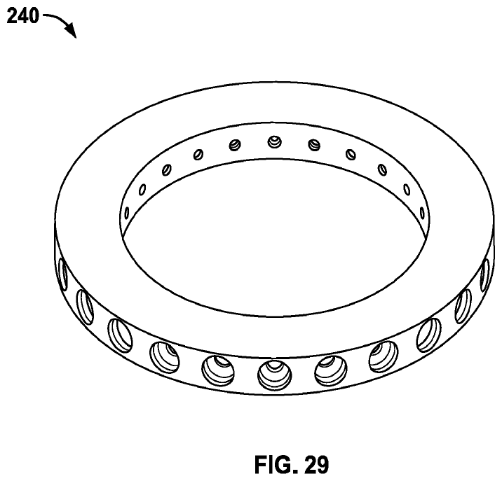

Referring to FIGS. 17A-C, a canted-coil spring 400, which is sized to grasp the crimp ring cap 410 of a culture bottle 230 is located at the distal end 415 of the bottle receptacle 220. The user pushes the culture bottle 230 into the bottle receptacle 220, thereby pushing the crimp 410 of the culture bottle past the canted coil spring 400, which holds the culture bottle 230 in the bottle receptacle 220. The bottom 416 of the culture bottle 230 extends out of the proximal end 420 of the bottle receptacle 220 so that the culture bottle bottom 416 can be grasped by the user or a robot for removal. FIGS. 17A-C are different views of the same mechanism. All are in the same cross-section. FIG. 17A is the entire bottle receptacle 220 with the culture bottle 230 therein. FIG. 17B is a detail plan view of the distal end 415 of the bottle receptacle 220 and FIG. 17C is a detail perspective view of the distal end 415 of the receptacle 220. One row of a drum formed from the receptacles illustrated in FIGS. 17A-C is illustrated in FIG. 29. The drum that is formed from such receptacles in faceted rather than completely circular.

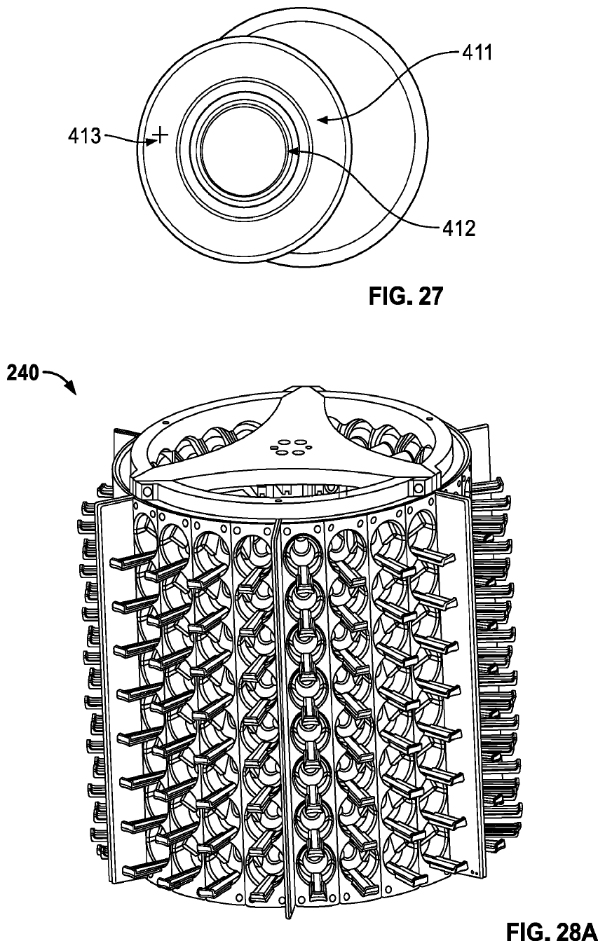

Optionally, the crimp cap 410 is a deformable metal (e.g. aluminum) cap with a septum in the center. With reference to FIG. 27, the top view of cap shows the metal crimp seal perimeter 411 with the metal crimp 412 which is recessed slightly from the metal crimp perimeter 411. The crimp cap 410 can carry a marking 413 that provides information about the status of the culture bottle (i.e. positive or negative for microbial growth) on which the cap is placed to the operator or user. To mark the crimp cap with a status indication, the blood culture bottle is pressed into a cavity that has at its bottom a raised symbol, in the illustrated embodiment a plus sign, to indicate that the system has determined that the culture bottle is positive for microbial growth. As illustrated, the marking 413 is aligned with the metal flat part of the crimp seal. When the cap is pressed on the raised symbol, an impression of the marking 413 is left on the crimp seal.

FIG. 29 illustrates a layer of a drum 240 with the receptacles illustrated in FIGS. 17A-C. The drum illustrated in FIG. 29 has faceted outer and inner perimeters due to the manner in which the receptacles fit together. The receptacles with the coil spring can be closely spaced vertically. Forming a drum with these receptacles can provide a significant height savings (e.g. about 50 mm). In embodiments where multiple drums are placed in a single cabinet, the cabinet size can be more compact and more easily accessible to users. Reduced drum height also allows more electronics to be placed in the top and bottom portions of the incubation and measurement cabinet. FIG. 29 illustrates one row of receptacles 220. As describe herein, a drum is assembled from multiple rows connected together to form the drum 240.

Because the culture bottle bottom 416 (FIG. 17A) extends from the receptacle 220, the culture bottle could obscure the station indicators on the proximal surface 463 of the receptacles 220 when viewed from or below the bottle receptacle. To address this potential issue, the station indicator light pipes (not shown) are extended from the distal end 415 of the receptacle to the proximal end 463 of the receptacle. Also, optionally, the culture bottle receptacles 220 could be sized so that the culture bottle fits completely therein, but wherein the proximal end of the receptacle is flared or indented adjacent the bottom of the culture bottles to provide the user or robot with grip access.

Another consideration is the critical nature of the position of the bottom of the bottle relative to its measurement optics and electronics. The canted coil spring must pull the bottle against a hard stop such that the bottle base does not move in any direction while the bottle drum is turning, or the bottle is touched by the user during manual workflow.

FIGS. 17D-F illustrate an alternative retention mechanism that has a ball plunger 474 disposed in the narrower portion 850 of the bottle receptacle 220. Referring to FIG. 17D, the ball plunger 474 has balls 471 biased outward by spring arm 476 and extending into the narrower portion 850 of the bottle receptacle 220. In FIG. 17D, the ball plunger 474 is illustrated from the distal end of the bottle receptacle 220, which is the end of the far end of the narrower portion 850 of the bottle receptacle 220. Referring to FIG. 17E, as the culture bottle 230 is advanced into the narrower portion 850 of the bottle receptacle 220, the balls 471 are seen extending outward into the narrower portion 850 of the bottle receptacle 220. Referring to FIG. 17F, as the culture bottle 230 is further advanced, the crimp ring cap 410 of the culture bottle 230 pushes the balls 471 back into passages 472 formed in the narrower portion 850 of the bottle receptacle 220. After the crimp ring cap 410 of a culture bottle 230 is advanced over the balls 471 of the ball plunger 470 the biasing force applied to the balls 471 causes them to advance into the space 472 formed between the crimp ring cap 410 of the culture bottle 230 and the neck 835 of the culture bottle 230. This biasing force secures the culture bottle 230 in the bottle receptacle 220. When the culture bottle 230 is removed from the bottle receptacle, the biasing force of the ball plunger 474 is overcome, allowing the crimp cap 410 of the culture bottle 230 to pass over the ball plunger 474 and be removed from the bottle receptacle 230.

FIG. 17G illustrates an alternative receptacle 220 wherein an O-ring 461 engages the detent 462 beneath the cap 410 of the culture bottle 230. Conventional o-rings are contemplated and can be made of materials such as Viton™, silicone or other conventional elastomeric materials. Such materials are well known to one skilled in the art and not described in detail herein. As illustrated in FIG. 17G, the receptacle 220 only receives a portion of the culture bottle 230 and terminates at 463 (about the transition from the neck 435 of the culture bottle to the bottom portion 416 of the culture bottle 230). That way it can be readily observed if the culture bottle does not seat properly in the receptacle, because the culture bottle will be askew from its desired position.

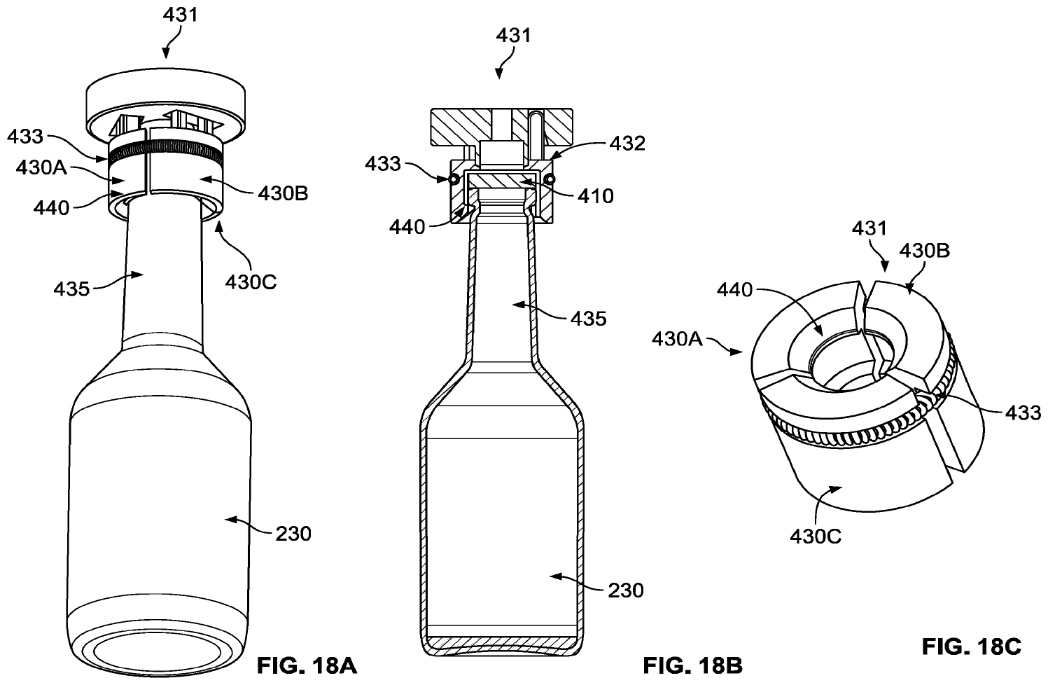

FIG. 18A-C illustrate a variation on the canted coil spring illustrated in FIG. 17. FIG. 18A is a perspective view of the retaining cap on the bottle. FIG. 18B is a cutaway view of the cap of FIG. 18A. FIG. 18C is an upside-down perspective view of the segmented retention mechanism of FIG. 18A. Referring to FIG. 18A, the retention mechanism 431 has multiple wedges 430A-C that translate radially to receive the neck 435 and crimp ring cap 410 (FIG. 18B) of the bottle 230. Three wedge sections 430A-C are illustrated, but a plurality of sections of a different number are contemplated. The wedge sections 430A-C are placed in the distal end of the narrower portion 850 of the culture bottle receptacle 220 in the drum. The crimp ring cap 410 attached to the neck 435 of the bottle 230 enters a flared proximal end 440 of the retention mechanism 431. The proximal end 440 of the retention mechanism 431 is flared to receive the crimp ring cap 410 of the bottle. The retention mechanism 431 has a stop flange 432 so that the bottle neck 435 can only be advanced a defined distance into the retention mechanism 431. The retention mechanism has a circumferential spring 433 that allows the wedges 430A-C to move radially in response to the bottle neck being inserted therein, but applies tension to ensure that the crimp ring cap 410 is held tightly in the retention mechanism.

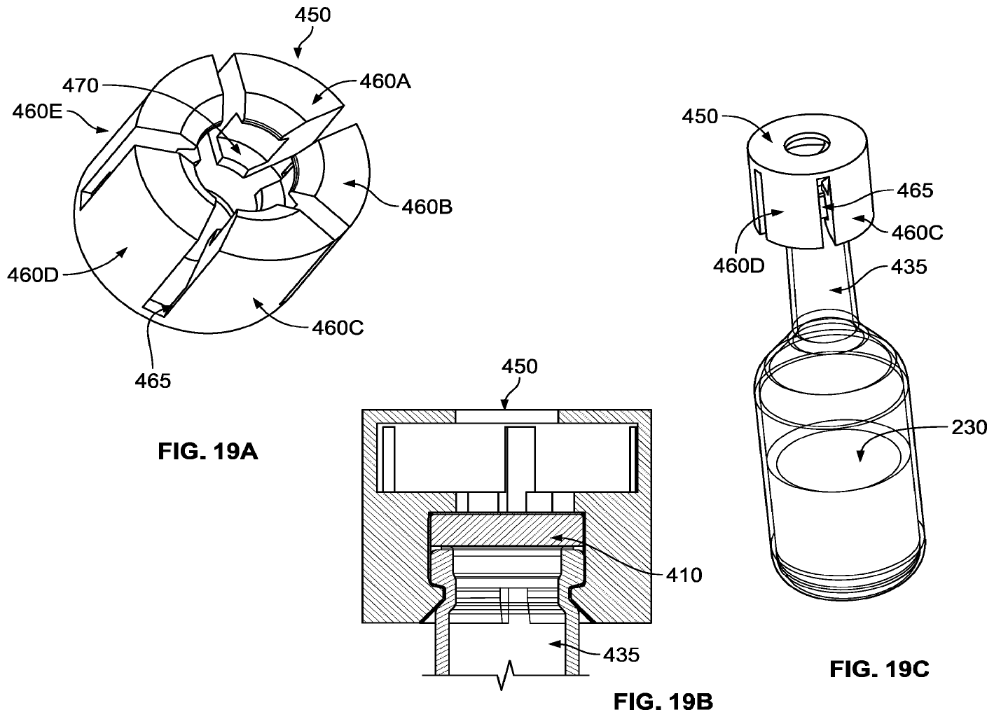

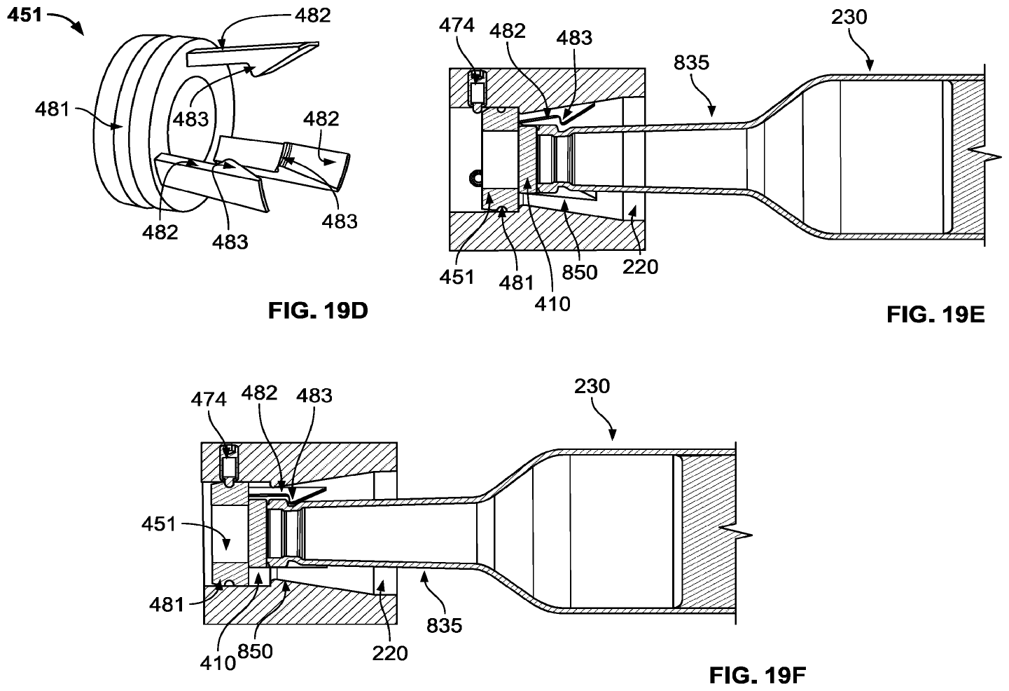

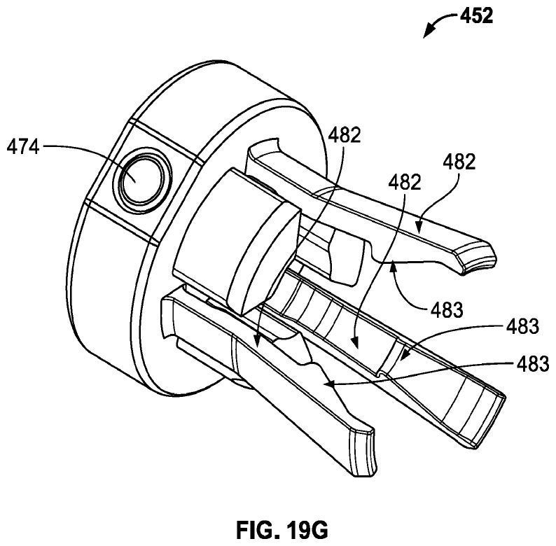

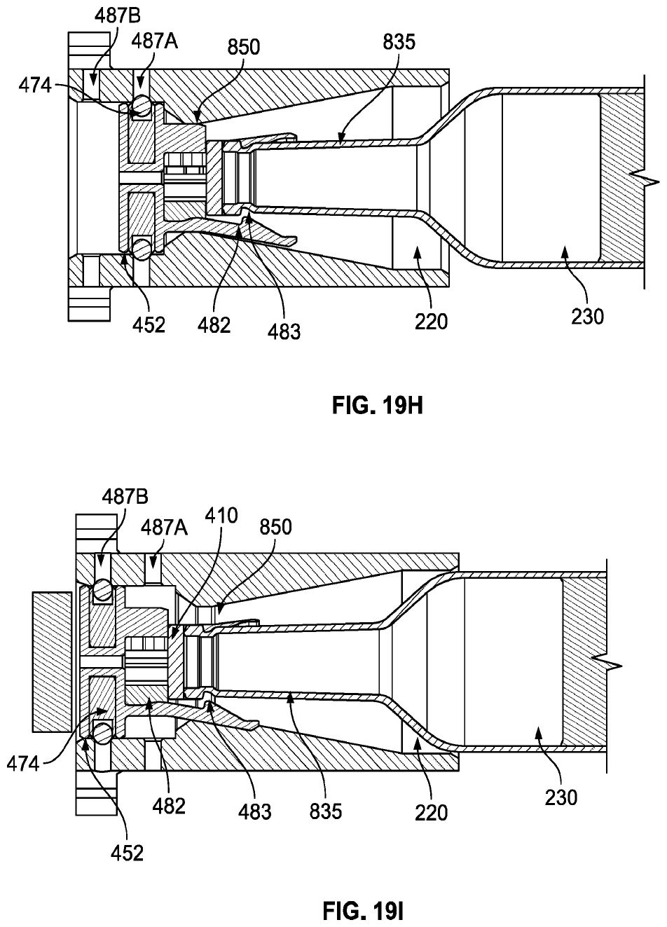

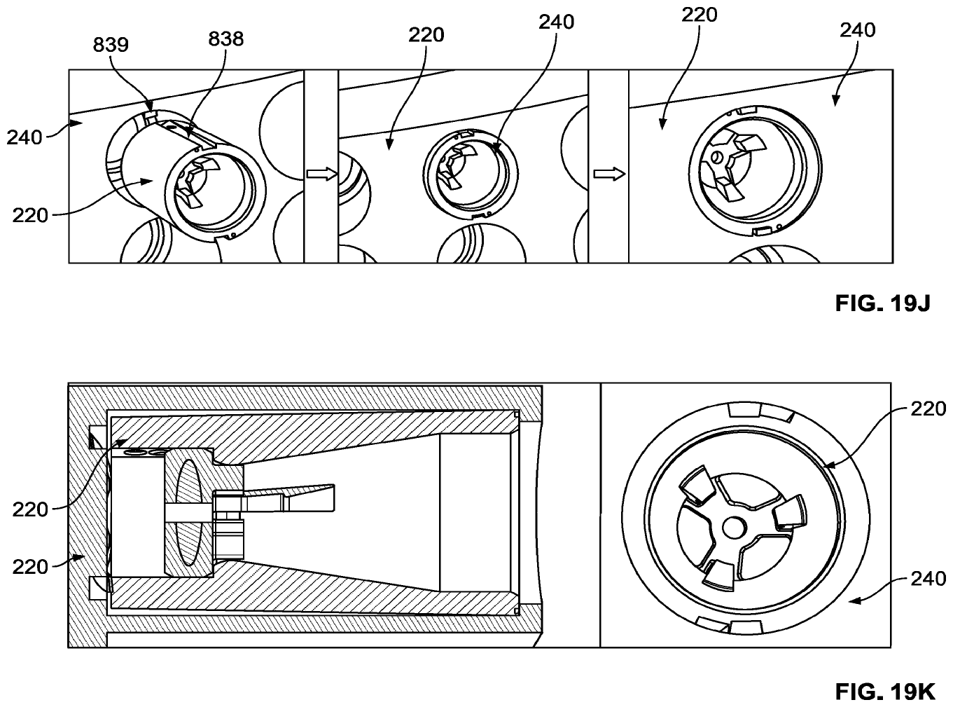

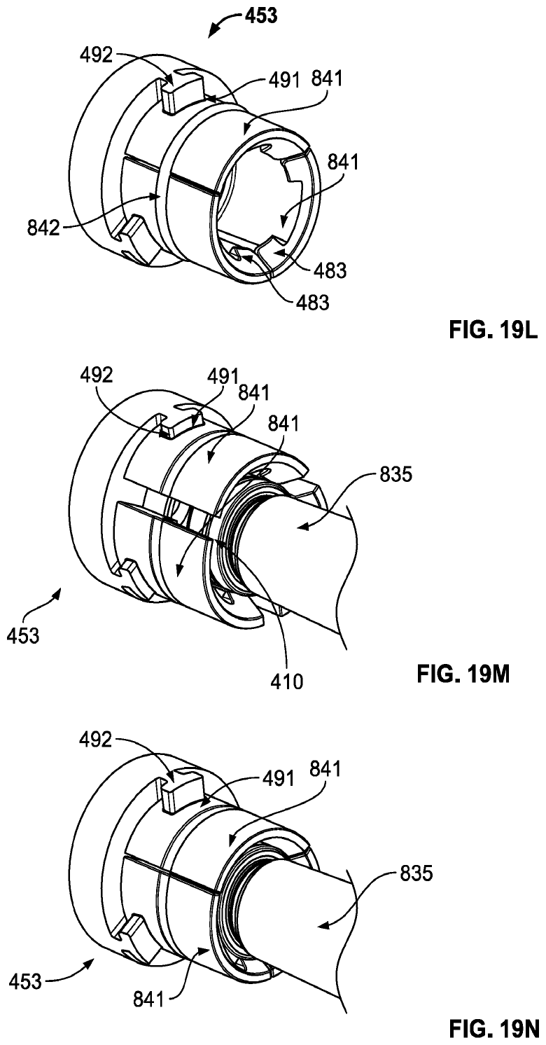

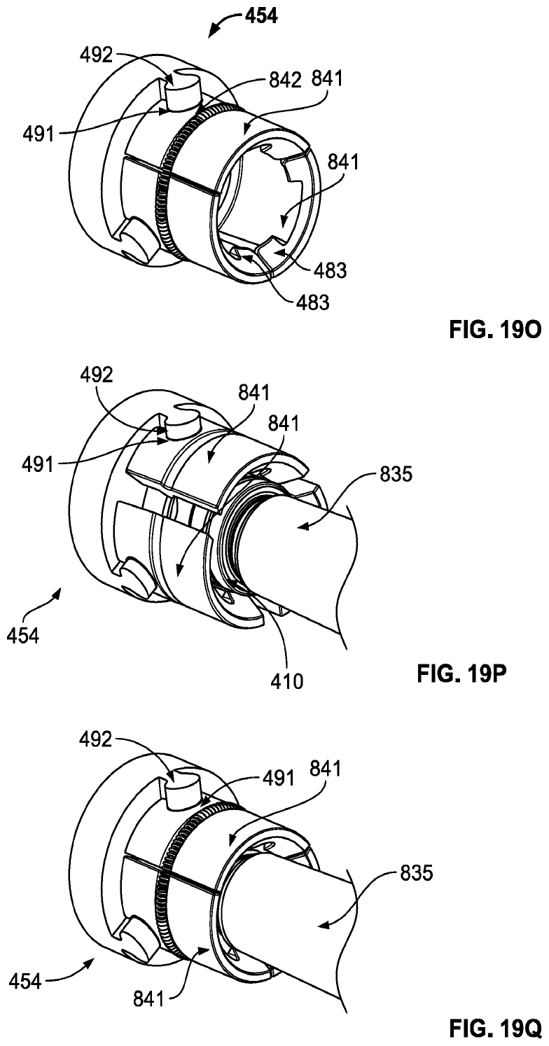

FIG. 19A-C illustrates a variation of the canted coil spring illustrated in FIG. 17. In this embodiment, the sectioned retention mechanism 450 has gripping sections 460A-E separated by spaces 465. Gripping sections 460A-E are resilient and spaces 465 allow the gripping sections to expand yet exert a gripping force on the crimp ring cap 410 and bottle neck 435 inserted therein. Like the embodiment in FIGS. 18A-C, the proximal end 470 of the retention mechanism 450 is flared to receive the crimp ring cap 410 and the bottle neck 435. In one example, the resilient gripping sections 460A-E are made of ABS plastic. Each section 460A-E bends radially to capture the crimp ring cap 410 and the bottle neck 435. Although five sections are illustrated in FIGS. 19A-C, this is for illustration. Retention mechanisms with a different number of sections are contemplated.

FIGS. 19D-Q illustrate other alternative retention mechanisms. FIG. 19D illustrates a slotted cap 451 that has a slot 481 that cooperates with ball plunger 474 to lock slotted cap 451 into the bottle receptacle 220. Wings 482 are tensioned and have flanges 483 that function much like second flanges 865 illustrated in FIG. 16B. Referring to FIG. 19E, as the culture bottle 230 is advanced forward in the bottle receptacle 220, the wings 482 are biased outward and receive the neck portion 835 of the culture bottle 230. The slot 481 of the slotted cap 451 is not aligned with the ball plunger 474 at this point. Referring to FIG. 19F, as the culture bottle 230 is further advanced toward the distal end of the bottle receptacle 220, the narrower inner portion 850 of the bottle receptacle 220 tapers, forcing the wings 482 inward such that flanges 483 fit in the space between the crimp cap 410 and the neck 835 of the culture bottle 230. Also, the slotted cap 451 is advanced such that the slot 481 aligns with balls 471 of the ball plunger, locking the slotted cap into place in the bottle receptacle. To remove the culture bottle, the biasing force of the ball plunger 474 is overcome, allowing the crimp cap 410 to pass over the ball plunger 474 so that the bottle can be removed. Since the wings 482 of the slotted cap 451 are biased outward, the flanges 483 remove out of engagement with the culture bottle 230 as the culture bottle is advance outward of the receptacle 220.

FIGS. 19G-I illustrate a ball plunger cap 452 that has a ball plunger 474 formed therein. The ball plunger cap 452 cooperates with slots 487A and 487B in the bottle receptacle 220 to lock ball plunger cap 452 into the bottle receptacle 220 when the ball plunger cap 452 is in the first unlocked position and the second locked position. Wings 482 are tensioned and have flanges 483 that function much like second flanges 865 illustrated in FIG. 16B. Referring to FIG. 19H, the cap 452 is in the first unlocked position and the ball plunger 474 is aligned with slot 487A. As the culture bottle 230 is advanced forward in the bottle receptacle 220, the wings 482 are biased outward and receive the neck portion 835 of the culture bottle 230. The slot 487B of the bottle receptacle 220 is not aligned with the ball plunger 474 at this point. Referring to FIG. 19F, as the culture bottle 230 is further advanced toward the distal end of the bottle receptacle 220, the narrower inner portion 850 of the bottle receptacle 220 tapers, forcing the wings 482 inward such that flanges 483 fit in the space between the crimp cap 410 and the neck 835 of the culture bottle 230. Also, the ball plunger cap 452 is advanced such that the slot 487A aligns with balls 471 of the ball plunger, thereby locking the ball plunger cap 452 into place in the bottle receptacle. To remove the culture bottle 230, the biasing force of the ball plunger 474 is overcome, allowing the assembly of the culture bottle 230 and the ball plunger cap 452 to advance to the first position, where the flanges 483 again bias outward and release the culture bottle 220. In the first position, slot 487A of the bottle receptacle 220 again aligns with ball plunger 474 of the ball plunger cap 452, thereby retaining the ball plunger cap 452 in the bottle receptacle when the bottle is removed from the culture bottle receptacle.

FIGS. 19J and 19K illustrate how the bottle receptacle 220 illustrated in FIGS. 19G-I fit into the drum 240. In FIG. 19J the bottle receptacle 220 has a slot 838 that is positioned in alignment with track 839 in the drum opening and advanced into the opening with this alignment until the bottle receptacle 220 is fully disposed in the opening of the drum 24. Referring to FIG. 19K, the bottle receptacle 220 is then rotated to lock the bottle receptacle 220 into the drum 240.

FIGS. 19L-19Q are alternative bottle receptacles to those illustrated in FIGS. 18A-C and FIGS. 19A-C. Referring to FIG. 19L, bottle holder 453 configured to be placed in a bottle receptacle has wings 841 and a canted coil spring 842. Other spring-like members such as an elastomeric o-ring or garter spring are also contemplated. The wings 841 can be forced apart, overcoming the bias of the canted coil spring 842 and illustrated in FIG. 19M as the neck 835 of the culture bottle is advanced into the bottle holder 453. When forced apart the wings 841 have a groove 491 that travels along track 492. Bottle holder 453 has flanges 483. When the crimp cap 410 of the culture bottle 230 is advanced past flanges 483, the biasing force of the canted coil spring 842 forces the wings back together and the flanges sit at the base of the crimp cap 410 securing the culture bottle in the bottle receptacle as illustrated in FIG. 19N. Again, the groove 491 in the wings 841 permit the wings 841 to travel back to their closed position along track 492. The canted coil spring is not illustrated in FIGS. 19M and 19N so that the travel of the wings from the forced open position in FIG. 19M to the closed position in FIG. 19N can be clearly seen.

FIG. 19O-Q illustrate a bottle holder 454 that is a variation of the bottle holder described in FIGS. 19L-N. Referring to FIG. 19O, bottle holder 454 configured to be placed in a bottle receptacle has wings 841 and a garter spring 842. The wings 841 can be forced apart, overcoming the bias of the garter spring 842 and illustrated in FIG. 19P as the neck 835 of the culture bottle is advanced into the bottle holder 454. When forced apart the wings 841 have a groove 491 that travels along track 492. Bottle holder 454 has flanges 483. When the crimp cap 410 of the culture bottle is advanced past flanges 483, the biasing force of the garter spring 842 forces the wings back together and the flanges sit at the base of the crimp cap 410 securing the culture bottle in the bottle receptacle as illustrated in FIG. 19Q. Again, the groove 491 in the wings 841 permit the wings 841 to travel back to their closed position along track 492. The garter spring is not illustrated in FIG. 19P so that the travel of the wings to the forced open position can be clearly seen.

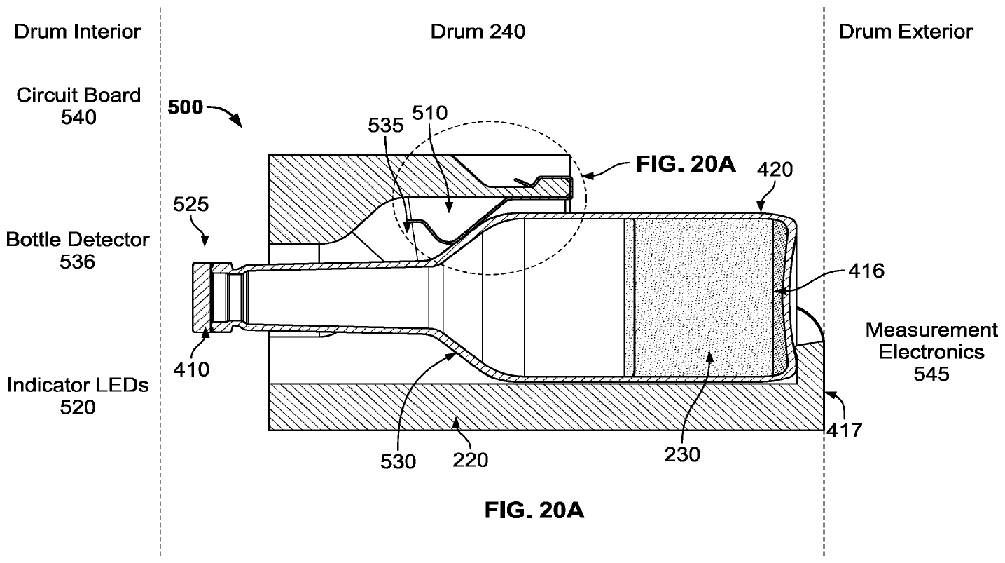

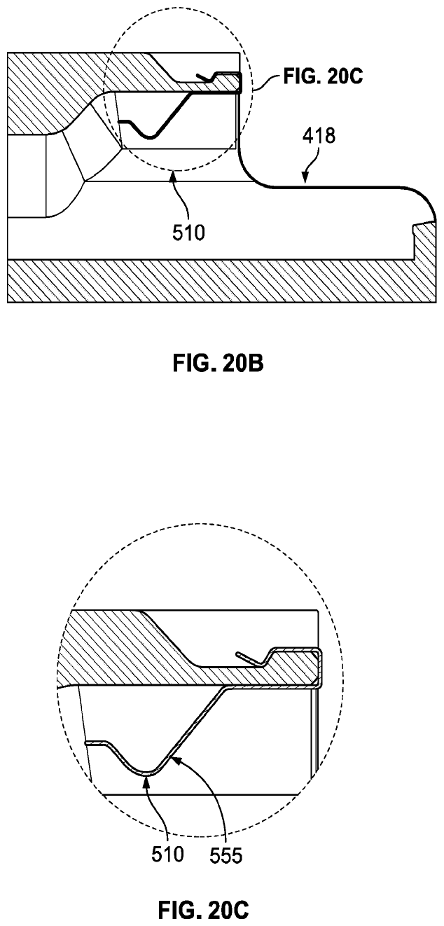

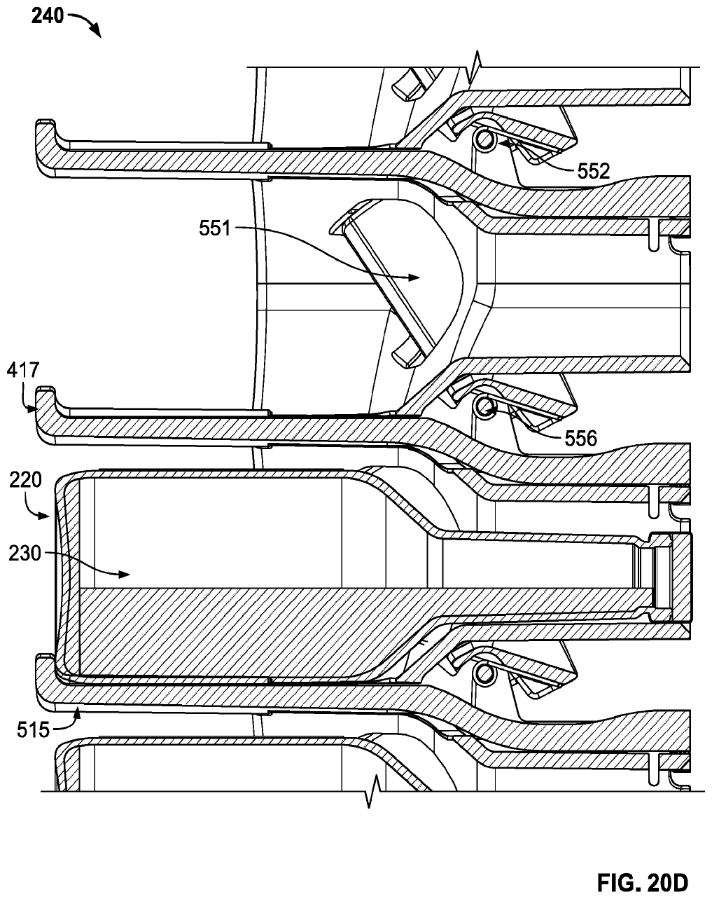

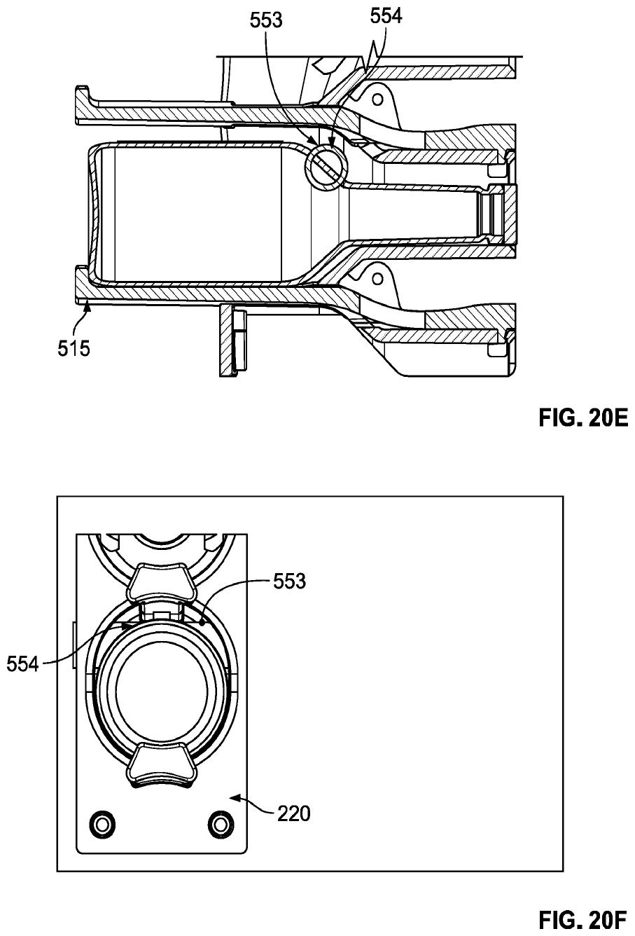

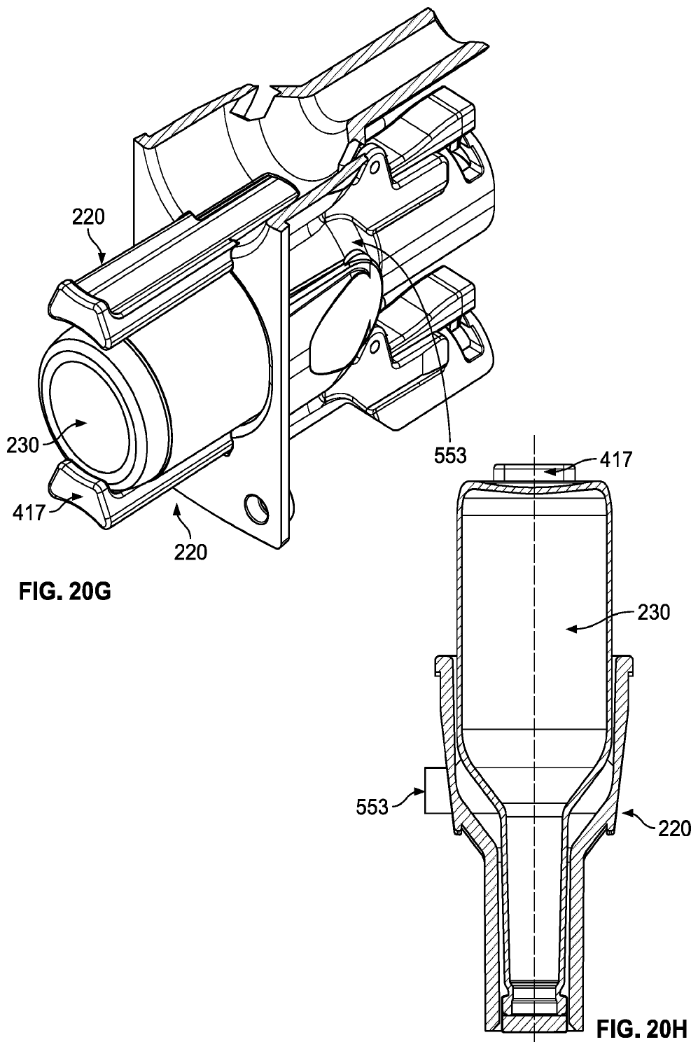

FIGS. 20A-C illustrate an alternate mechanism 500 for retaining the bottle in the drum. The mechanism 500 uses a flat spring 510 to secure the culture bottle 230 in the receptacle 220. In this configuration, the culture bottle bottom 416 is secured in the receptacle by tab 417. The holder 220 has opening 418 such that the culture bottle 230 may be inserted and removed from the holder 220 either manually or by being gripped by a robot. This permits access to the culture bottle 230 secured in the drum receptacle 220. The user or robot pushes the bottle into the receptacle 220 until the flat spring 510 presses on the bottle and the bottle bottom 416 drops in front of the tab 417. The flat spring 510 holds the bottle against stop 417. To remove the culture bottle 230, the user or robot presses the culture bottle 230 away from the tab 417 to allow the bottle 230 to be withdrawn from the receptacle 220. When the culture bottle 230 is not secured by the tab 417, the flat spring 510 also pushes the culture bottle 230 toward the proximal end 420 of the receptacle 220 for ease of retrieval.

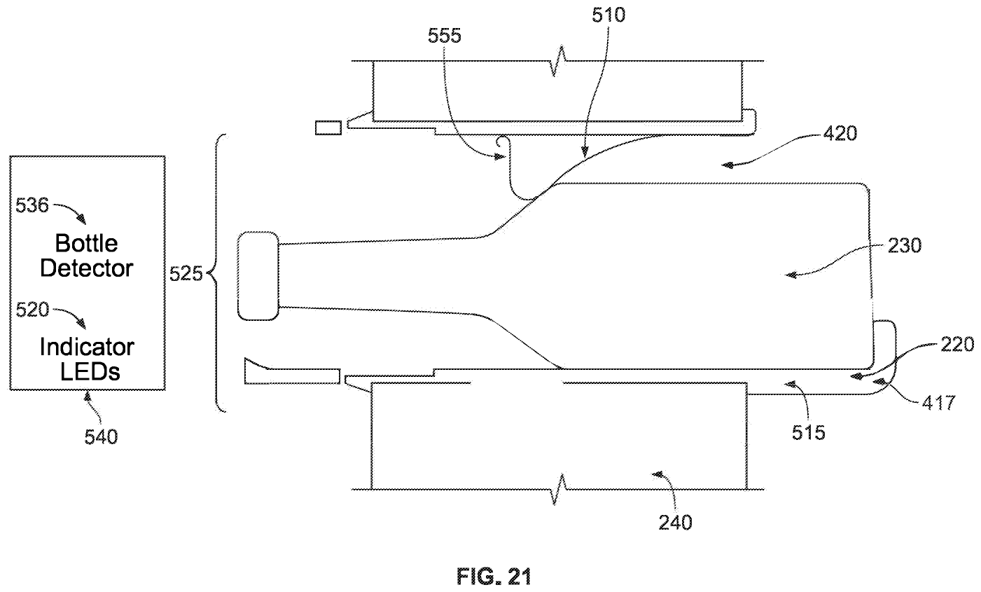

With reference to FIG. 21, the receptacle also has a light pipe 515 to translate the light from indicator LEDs 520 from the distal end 525 of the receptacle (i.e. the inside of the drum 240 in which the receptacle is disposed). The light pipe 515, if present, cradles the bottle 230 and extends past the proximal end 420 of the receptacle (i.e. the outer surface of the drum 240). The flat spring 510 with free end 535 presses against the upper shoulder 530 of the culture bottle 230 to hold the culture bottle 230 against the tab 417. The receptacle 220 is adjacent to a bottle detector 536 at the distal end 525 of the receptacle 220. The distal end of a bottle 230 in receptacle 220 is detected by bottle detector 536. The bottle detector 536 is carried by the stationary detector board 540. As illustrated in FIGS. 20B and 20C, the spring is enmolded into the bottle holder 220.

The module contains measurement electronics 545 for each layer of bottles 230 in each drum 240. As illustrated in FIG. 20A, the measurement electronics 545 are placed on the outside of the drum 240 in the front right corner of the module 210 (see FIGS. 4 and 10).

The light pipe 515 (FIG. 21) lines up with indicator LEDs 520 on the board 540. The surface of the end of the light pipe 515 outside the bottle drum 240 is textured to disperse the light from the indicator LEDs 520. The bottle crimp cap 410 interrupts the bottle detector 536 (e.g., an optical switch or a proximity sensor) when it is placed in the receptacle 220. The indicator LEDs 520 and bottle detector 536 are located on boards 540 located on the inside of the drum 240 in positions that correspond to each bottle 230 in the drum 240 that is accessible by the user. The bottle detectors 536 are monitored while a door to the module is open to detect in real time when a bottle 230 is placed in a receptacle or removed from a receptacle.

Referring to FIG. 20A. the user places a bottle 230 into the receptacle 220 by inserting the bottle 230 neck first until it contacts the flat spring 510, then pushing the bottle 230 against the spring 510 until the culture bottle bottom 416 slips by the tab 417 that holds the bottle 230 in the receptacle 220. The flat spring 510 engages the culture bottle 230 such that, if the user does not fully enter the culture bottle 230 into the receptacle 220, the flat spring 510 will prevent the culture bottle 230 from fully seating in the receptacle 220. As a result, the culture bottle 230 will extend far enough out of the station so that the drum 240 will not be able to advance the culture bottle 230 past the module housing 224. This will allow the module 210 to detect unseated bottles 230 once the user closes the module door. To remove a bottle 230 from a receptacle 220, the user simply lifts the culture bottle bottom 415 over the tab 417 and pulls the culture bottle 230 out of the receptacle 220. In those embodiments where insertion and removal of the culture bottles is automated, the same motions are performed by an automated apparatus.

Referring to FIG. 20C, the receptacle 220 is illustrated in greater detail with a few modifications. The receptacle 220 is made of a clear material so it can be used as the light pipe to transfer light from the receptacle status indicator LEDs 520 inside the drum 240 to the proximal end 420 of the receptacle 220 on the outside of the bottle drum 240.

The flat spring 510 that is attached to the top of the receptacle 220 bends from the position illustrated in FIG. 20C to the position illustrated in FIG. 20A after the culture bottle 230 is received into the receptacle 220. When a free end 555 of the flat spring 510 contacts the top of the receptacle 220 the spring 510 becomes a hard stop and prevents the culture bottle 230 from moving farther into the receptacle 220. When the culture bottle 230 is released after insertion the flat spring 510 pushes the culture bottle 230 against the tab 417 of the receptacle 220 that extends out from the outer surface of the drum 240. The culture bottle 230 is removed from the receptacle 220 by lifting the bottom 416 of the culture bottle 230 up through the proximal end 420 of the receptacle 220 either manually or using a robot.

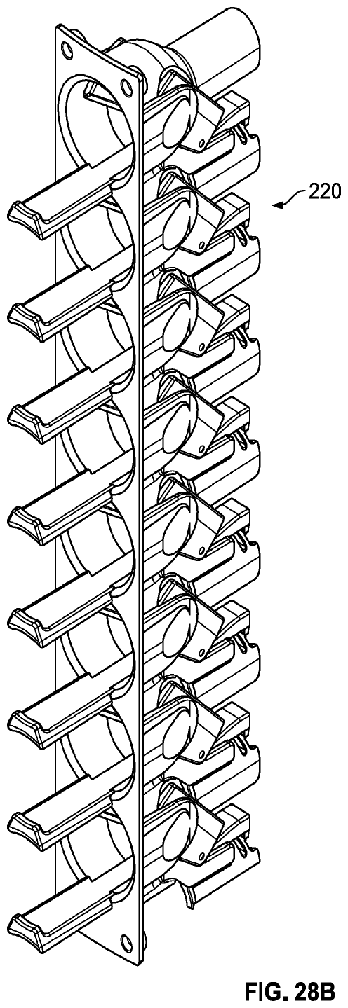

FIG. 28A illustrates a drum 240 with the receptacles illustrated in FIGS. 20A-C and 28B. The receptacles 220 with the flat spring are provided as a column and the drum 240 is formed by attaching the columns together. The resulting structure is a round drum 240 (FIG. 29A) with circular exterior and interior perimeters because of the way in which the rows fit together.

FIG. 20D illustrates, a portion of a drum 240, with multiple vertical rows of the receptacles 220 illustrated in FIGS. 20A-C. The drum is illustrated in cutaway view to show the culture bottle 230 support in receptacles 220. The top receptacle 220 is empty. In the embodiment illustrated in FIG. 20D a pivoting arm 551 is provided to secure the culture bottle 230 in the receptacle 220 instead of the leaf spring 550 previously described. As the bottle 230 is advanced into the receptacle 230 the pivoting arm 551 rotates clockwise to secure the culture bottle 230 in the receptacle. Resistance to pivoting arm 551 is applied by coil spring 552 which is secured in the receptacle with a pin 556.

An alternative to the receptacle illustrated in FIG. 20D is illustrated in FIGS. 20E-H. Referring to FIG. 20E, the pivoting arm 551 illustrated in FIG. 20D is replaced by a deformable material 553. In one example, the deformable material 553 is a peristaltic tubing but other conventional deformable materials are contemplated. A key aspect of the deformable material is its resilience in assuming its undeformed shape after each instance of being deformed by insertion of the culture bottles in the receptacle. The bottom portion of the receptacle 220 is light pipe 515.

The deformable material 553 is placed in tapered portion 554 of the receptacle 220. Referring to FIG. 20F, an end view of the receptacle (220) illustrates the deformable material 553 at the top portion of the receptacle, (along the tapered portion 554 of the receptacle). Suitable deformable materials include, in addition to the elastomeric peristaltic tubing described above, elastomeric materials and foam materials.

FIG. 20G is perspective view of one receptacle 220 with a portion of second receptacle formed above it. The culture bottle is retained in the receptacle as described above. Tab 417 secure retains the culture bottle 230 in the receptacle 220. Other materials of deformable material are contemplated as having sufficient frictional properties when used in contact with bottle 230. Such friction prevents the rotation of bottle 230 thus allowing the measurement system to obtain high quality signals with less noise caused by rotationally vibrating and rotating bottle.

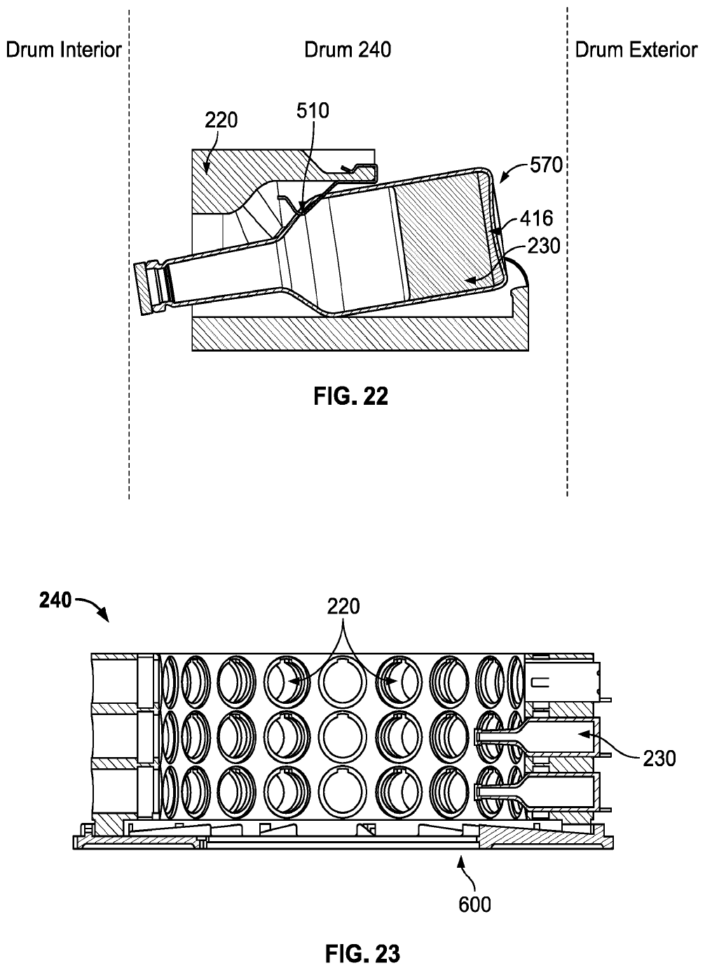

Referring to FIG. 22, if the bottle is not fully inserted into the receptacle 220, the flat spring 510 will hold the bottle in the overlay position 570. That way, the culture bottle 230 cannot appear to be properly seated in the receptacle 220 when it is not. When a culture bottle 230 is in the receptacle but not fully seated therein, the bottom 416 of the culture bottle will not allow the bottle drum 240 to be rotated by the module. The module detects the unseated bottle 570 by detecting the jammed bottle drum 240 and sends a signal to the system. The user will be notified to clear the obstruction by removing or fully inserting the bottle 230.

The neck-in orientation of the culture bottles offers some improvements over the neck out orientation of the prior art. Prior art drums held the culture bottles at an angle of 20° above horizontal during readings. With the bottle necks pointed inwardly, rotating the bottle drum forces the culture medium/resin in the bottles towards the bottom of the bottle. Therefore, in the neck-in configuration, the culture bottle can be held in the drum in a horizontal position. During the optical interrogation of the bottle, the drum spins at a speed that forces the culture medium/resin in the culture bottles to the standard 20° angle relative to the side of the culture bottle. For a drum that locates the bottle bottoms at a diameter of 22 inches. The drum diameter is measured from the bottom of the bottle on one side of the drum to the bottom of the culture bottle on the opposite side of the drum. A drum rotation speed of approximately 37 RPM induces a 20° angle for the culture medium/resin toward the bottom of the bottles. For comparison, the rotor in prior art apparatus spins at about 30 RPM for both reading the culture bottles and for agitation. Optionally, a drum rotation speed could be selected to cause the drum to operate like a centrifuge, driving additional separation of the solid portions of the sample (i.e. the culture medium and resin) from the liquid portions of the sample.

FIG. 26 illustrates a bottle stop 1000 that can be placed at the end of bottle receptacle 220. The bottle stop 1000 is a simple keyhole slot 1010 that has a top opening 1015 with a circumference sufficient so that the crimp cap of the culture bottle passes therethrough. As the culture bottle seats in the receptacle 220, the neck of the culture bottle settles into the narrower opening 1020 of the keyhole slot 1010, which is not wide enough for the crimp cap of the culture bottle to pass therethrough. Consequently, the keyhole slot 1010 retains the culture bottle in place in the bottle receptacle until the culture bottle is removed therefrom either manually or by use of automation (e.g. a robot).

The orientation of the culture bottles with their necks in requires the measurement electronics to be located on the perimeter of the bottle drum. As illustrated herein, these electronics are positioned in the front right corner. The culture bottle bottoms are spaced further apart in the neck in configuration than in the neck out configuration. This reduces any possible cross-talk for the measurement system (e.g., cross-talk from fluorescence emission from a bottle adjacent to the bottle being measured is reduced or eliminated).

As explained herein, the module rotates the drum both for positioning the bottles for user access and also for automation access. The module also rotates the culture bottles to agitate them.

Optionally, the drum motion system has a direct drive motor attached to an axle on which the drum bearings are mounted, and the drum rotates around the axle. Direct drive motors do not require the additional parts that would be required for belt drive or rim drive motors. The direct drive motors are a more reliable solution and make less noise than other conventional motors.

To agitate the bottles in a drum in which the bottles are held neck-out, the drum is accelerated to force the culture medium/resin into the neck of the bottle using centrifugal force. The drum is then decelerated to allow the culture medium/resin to flow back to the bottom of the culture bottle. Optionally, this motion profile is repeated about every two seconds to create motion of the culture medium/resin in the bottles that mimics the motion of the culture medium/resin when the culture bottles are rocked in prior art apparatus.

The horizontal acceleration of the bottle that occur during the angular acceleration agitation process describe above does not occur in prior art apparatus and the agitation that results is advantageous. Optionally, the drum will be operated to provide low frequency acceleration and deceleration through a large angle of the drum. Such motion will move the liquid in the culture bottle (i.e. the culture medium/resin/sample) from the base of the culture bottle to its neck and back.

Optionally, the drum will be operated to provide high frequency acceleration and deceleration through a small angle of the drum. Such motion moves the liquid in the culture bottle (i.e. the culture medium/resin/sample) from side to side in the bottle. This motion is similar to the motion imparted to the liquid in the culture bottle by prior art apparatus.

Optionally, variations and combinations of the above motion profiles, with the drum moving back and forth, or in a single direction, are contemplated.

Optionally, the drum is operated to impart periodic abrupt deceleration to dislodge blood, resin, and bacteria that may have settled in the bottle. Some bacteria would be harmed with such continuous violent agitation. Such motion is infrequent during instrument operation, (e.g. once an hour or once at the end of each measurement cycle).

Other mechanisms for moving the drum instead of the direct drive motor and bearings on a solid axle described above are contemplated. The objectives of such alternative mechanisms are: i) minimize the vertical space between bottle drums; minimize the space between drums to increase the bottle density in the cabinet; and iii) provide a more uniform column of bottles to the user during manual workflow.

One example is a carousel mounted to gear adaptor ring (GAR) that turns on a ring bearing, and is driven by a motor and toothed belt. The bottle drum in the module could be similarly mounted to a GAR and driven by a toothed belt. The bottle drum would be open at the top and all connections to the electronics inside the drum would be routed above the drum.

FIG. 23 illustrates such a drum 240, which rotates on a large diameter ring bearing 600. The curvature of the drum 240 is apparent from the variation in distance and angle of bottle receptacles 220, Bottles 220 are neck in, which means the measurement system (not shown) resides outside the drum assembly. The illustrated embodiment accommodates a belt driven base by a motor outside of the drum. Optionally, a direct drive motor located in the center of the base and ring bearing could be used. The measurement system in the modules described herein are no more sensitive to the speed of the bottle drum than the sensitivity of measurement systems in the prior art systems to the rotor speed.

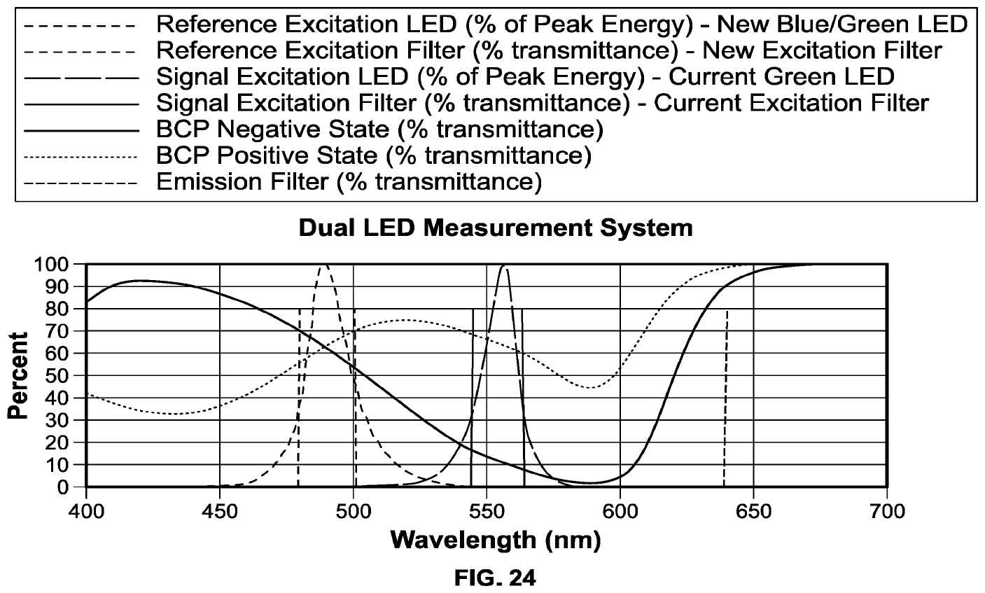

Referring to FIG. 24, the apparatus described herein optionally has LEDs either having a first color (e.g. green) or a second color (e.g. blue) for source illumination. The additional LED obtains a reference reading. As seen in FIG. 24, the light indicating the reference reading is activated when the indicator dye (bromocresol Purple (BCP) in the positive state (% transmittance) is equal to BCP in the negative state. A positive indication is obtained when the percent transmittance for the positive state is in excess of the % transmittance for the negative state. The wavelength of the blue LED is at a point on the response curve that is not affected by the pH state of the blood culture in the culture bottle. The ratio of the reading from the bottle using the blue excitation light and the reading obtained with the green excitation light should be proportional to the slope of the BCP response curve, which is proportional to the pH state of the BCP. A pH reading from the BACTEC bottles is an improved indicator of the biological growth in the bottle.

The apparatus described herein provides the following advantages: 1) a reduction of noise (i.e., the ratio of growth signal to reference signal should be unaffected by bottle position, temperature, and sensor variability); 2) detection of growth in a vial that experiences a delay in entry into the system (i.e. the dual measurements described above provide a reference such that the contents of the vial do not need to be sampled continuously during growth to confirm positivity by detecting growth acceleration); and 3) signal quality indicator (i.e., the reference signal is an independent indicator of the health of the station hardware).

Also described herein is a method for operating the apparatus described herein. According to the method the motion of the bottle drum is controlled to support a user workflow. The user requests that the module door be opened to access a bottle drum containing blood culture bottles as illustrated herein. The bottle drum is stopped by the bottle drum drive motor at a specific position prior to the door opening. As described above, only a sector of the bottle drum is accessible to a user when the module door is opened. The user can indicate to the control system which sector of the drum the user wishes to access.

Once the door is opened, a door interlock switch will reduce the maximum power that is delivered to the bottle drum motor. In this way, the bottle drum motor drives the bottle drum at low power and low speed. The status of the bottles in the bottle drum are indicated with lights near each culture bottle receptacle in the bottle drum. As described herein, the light sources and sensors are in fixed positions inside the bottle drum module. The light sources illuminate a light pipe for a station in the bottle drum when that station is aligned with the light source.

The user accesses the bottle drum to insert culture bottles in available stations (also referred to herein a bottle receptacles), or remove negative and positive bottles from stations. The user removes culture bottles with a target status (i.e., positive or negative). For example, positive bottles are accessed by the user for further work-up; negative bottles are collected by the user for disposal. Once the user access to the accessible section of the drum is complete, the user is permitted to manually move the drum left or right, overcoming the small resistance to rotation exerted by the motor. The change in position of the drum is sensed by the control system and effectively the force exerted by the motor is removed, allowing the drum to turn relatively freely. Once the control system releases the drum from the motor resistance to manual rotation, the control computer determines the next position of the bottle drum that will permit the user to access the culture bottles of interest. When the culture bottles of interest are accessible to the user, the control system sends a signal to the bottle drum motor to stop the bottle drum in that position.

As the bottle drum turns, the bottle status indicator light sources are illuminated such that the status of a culture bottle is correctly indicated via the light pipe aligned with a bottle receptacle/station. The bottle drum control system includes an encoder that communicates with the bottle drum control system so that the control system is aware of the position of the bottle drum at all times (and therefor the position of each receptacle/station at all times). The control system illuminates each station/receptacle as the bottle drum advances the bottle receptacle/station advances from one location to the next.

In the methods described above, it may not be desirable for the user to advance the drum or carousel manually. Contemplated herein are systems with controls that allow the user to advance rotation of the drum to reveal additional bottles. Such controls can simply be buttons or icons on a touch panel that allow the user to advance the drum in the clockwise or counter clockwise direction. If the system described herein is equipped with such controls, the user is prevented from overriding those controls and moving the drum manually. Such controls can be used to advance the drum or carousel incrementally (i.e., intermittent actuation) or continuously (by sustained actuation).

In this specification, the word “comprising” is to be understood in its “open” sense, that is, in the sense of “including”, and thus not limited to its “closed” sense, that is the sense of “consisting only of”. A corresponding meaning is to be attributed to the corresponding words “comprise”, “comprised” and “comprises” where they appear.

While particular embodiments of this technology have been described, it will be evident to those skilled in the art that the present technology may be embodied in other specific forms without departing from the essential characteristics thereof. The present embodiments and examples are therefore to be considered in all respects as illustrative and not restrictive.

It will further be understood that any reference herein to subject matter known in the field does not, unless the contrary indication appears, constitute an admission that such subject matter is commonly known by those skilled in the art to which the present technology relates.

1. An apparatus for storing and monitoring blood culture bottles, the apparatus comprising:
a drum having an exterior perimeter and an interior perimeter, the exterior perimeter having a diameter in excess of a diameter of the interior perimeter, the drum having a plurality of receptacles, the receptacles having a proximal end at the exterior perimeter and a distal end at the interior perimeter, each receptacle configured to receive a blood culture bottle, the blood culture bottle comprising a bottom portion and a neck portion, wherein the bottle can be received by the receptacle such that either the bottom portion is received at the distal end of the receptacle or the neck portion is received at the distal end of the receptacle;
wherein the drum perimeters are disposed about an axis of rotation of the drum;
wherein the plurality of receptacles are disposed in the drum as an array of receptacles, the array having receptacles disposed both vertically and horizontally;
sensors and detectors for interrogating the blood culture bottles to determine if the blood culture bottles are positive or negative for microbial growth; and
the drum defining an interior space within the interior perimeter wherein at least a portion of drum electronics in communication with the sensors and detectors for interrogating the blood culture bottles are disposed in the interior perimeter of the drum.

2. The apparatus of claim 1, wherein a plurality of drums are disposed in a housing.

3. The apparatus of claim 2, wherein the housing has a door to access each drum disposed in the housing.

4. The apparatus of claim 2, wherein the housing has a touch screen.

5. The apparatus of claim 2, wherein the housing further comprises a rack for receiving culture bottles to be placed in or removed from the apparatus.

6. The apparatus of claim 3, wherein the drum has a plurality of panels that define vertical drum sectors.

7. The apparatus of claim 6, wherein each vertical drum sector has an approximately equal horizontal span and each span is such that the horizontal span of one vertical drum sector is approximately the same as a horizontal span of the door.

8. The apparatus of claim 2, wherein the apparatus further comprises a drive motor for rotating the drum disposed in the housing.

9. The apparatus of claim 1, wherein measurement electronics are disposed within the interior space defined by the interior perimeter of the drum.

10. The apparatus of claim 1, wherein culture bottle indicator electronics are disposed within the interior space defined by the interior perimeter of the drum.

11. The apparatus of claim 2, further comprising a heater disposed in the housing outside the exterior perimeter of the drum.

12. The apparatus of claim 2, further comprising a blower disposed in the housing outside the exterior perimeter of the drum.

13. The apparatus of claim 8, further comprising an axle that defines the axis of rotation and at least one bearing that is disposed coaxially with the axle and the drum to allow the drum to rotate about the axle.

14. The apparatus of claim 13, wherein the bearing is a ring bearing.

15. The apparatus of claim 1, wherein the drum is assembled in layers, each layer being a at least a portion of an entire row of receptacles and each layer having the interior perimeter and the exterior perimeter.

16. The apparatus of claim 1, wherein the drum is assembled in column segments, each column segment defining only a portion of the interior perimeter and the exterior perimeter of the drum.

17. The apparatus of claim 1, wherein the plurality of receptacles each have a respective elastomeric insert for receiving a culture bottle.

18. The apparatus of claim 1, wherein the plurality of receptacles each receive the neck portion of the culture bottle at the distal end and each receptacle comprises a bottle stop and a canted coil spring spaced apart from the bottle stop, the canted coil spring biased to permit a cap disposed on the neck portion of the bottle to pass by the canted coil spring and proceed to rest against the bottle stop, whereby the canted coil spring releases tension and secures the culture bottle in the receptacle.

19. The apparatus of claim 1, wherein the plurality of receptacles each receive the neck portion of the culture bottle at the distal end and each receptacle comprises a bottle stop and an o-ring spaced apart from the bottle stop, the o-ring is sufficiently elastic to permit a cap disposed on the neck portion of the bottle to pass by the o-ring and proceed to rest against the bottle stop, whereby the o-ring then releases tension and secures the culture bottle in the receptacle.

20. The apparatus of claim 1, wherein the plurality of receptacles each receive the neck portion of the culture bottle at the distal end and each receptacle comprises a bottle stop and a ball plunger spaced apart from the bottle stop, the ball plunger biased to permit a cap disposed on the neck portion of the bottle to pass by the ball plunger and proceed to rest against the bottle stop, whereby the ball plunger releases tension and secures the culture bottle in the receptacle.

21. The apparatus of claim 1, wherein the plurality of receptacles each receive the neck portion of the culture bottle at the distal end and each receptacle comprises a bottle stop and a segmented retention portion spaced apart from the bottle stop, the segmented retention portion biased to permit a cap disposed on the neck portion of the bottle to pass into the segmented retention portion and proceed to rest against the bottle stop, whereby the segmented retention portion releases tension and secures the culture bottle in the receptacle.

22. The apparatus of claim 21, wherein the segmented retention portions are urged together by a canted coil spring.

23. The apparatus of claim 21, wherein the segmented retention portions are resilient segments.

24. The apparatus of claim 1, wherein the plurality of receptacles each receive the neck portion at the distal end and each receptacle comprises a bottle stop and a plurality of resilient wings extending from the bottle stop, the wings biased to permit a cap disposed on the neck portion of the bottle to pass past a flange portion of the wings and proceed to rest against the bottle stop, whereby the wings release tension and the flange portion secures the culture bottle in the receptacle.

25. The apparatus of claim 24, wherein the plurality of receptacles each further comprise a ball plunger and the bottle stop comprises a notch, wherein, as the culture bottle advances into the bottle stop, the bottle stop is advanced further into the receptacle until the ball plunger aligns with the notch, thereby securing the bottle stop in the receptacle.

26. The apparatus of claim 24, wherein the plurality of receptacles each further comprise a notch and the bottle stop comprises a ball plunger, wherein, as the culture bottle advances into the bottle stop, the bottle stop is advanced further into the receptacle until the ball plunger aligns with the notch, thereby securing the bottle stop in the receptacle.

27. The apparatus of claim 1, wherein the plurality of receptacles each further comprises a tray portion on which the culture bottle is placed, the tray portion comprising a tab for securing the bottom of the culture bottle in the tray the receptacle further comprising a stop portion, wherein the stop portion places the culture bottle in a predetermined fixed position within the receptacle.

28. The apparatus of claim 27, wherein the stop portion comprising a leaf spring.

29. The apparatus of claim 27, wherein the stop portion comprises a deformable material.

30. The apparatus of claim 29, wherein the stop portion further comprises a pivoting arm that pivots in response to the culture bottle being advanced into the receptacle, thereby securing the culture bottle in the receptacle.

31. The apparatus of claim 27, wherein the stop portion comprises a resilient material selected from one of elastomeric flexible tubing, elastomeric materials, or foam materials.

32. The apparatus of claim 27, wherein the tray portion further comprises a light pipe that transmits a light signal from indicator LEDs disposed within the inner perimeter of the drum to a light detector positioned on the exterior of the drum.

33. The apparatus of claim 27, wherein the bottle stop comprises a keyhole element.

34. (canceled)

35. The apparatus of claim 1, wherein the exterior perimeter of the drum and the interior perimeter of the drum are circular.

36. The apparatus of claim 1, wherein the exterior perimeter of the drum and the interior of the drum are faceted.

37. A method for controlling an incubator for a plurality of blood culture bottles, the method comprising:
inputting, via a control interface, a predetermined sector of a blood culture drum, the blood culture drum comprising a rack that defines a substantially circular interior perimeter and a substantially circular exterior perimeter wherein the blood culture drum is a rotating drum;
opening a door of a housing for the blood culture drum;
substantially stopping the drum by reducing power to a motor used to rotate the drum such that the predetermined sector is accessible via the opened door of the housing;
wherein the blood culture drum comprises a plurality of receptacles, each receptacle of the plurality of receptacles adapted to receive and retain a blood culture bottle, wherein the blood culture bottle is shaped to include a neck portion, wherein each receptacle of the plurality of receptacles has an indicator at a proximal end thereof, the indicator providing an indication that a culture bottle in the receptacle is positive or negative for microbial growth; and
at least one of inserting a blood culture bottle into an empty receptacle of the plurality of receptacles, removing a blood culture bottle from a receptacle of the plurality of receptacles, or removing a blood culture bottle from one receptacle of the plurality of receptacles and inserting, in its place, a different blood culture bottle wherein the blood culture bottle is removed based on its indicated status.

38. The method of claim 37, wherein the receptacles each further comprise a light pipe extending from the proximal end of the receptacle to a distal end of the receptacle.

39. The method of claim 37, wherein the control interface is in communication with an encoder such that the control interface tracks a placement of each blood culture bottle in the rack.

40. The method of claim 38, wherein the incubator further comprises a reader station, wherein the reader station determines the status of the blood culture bottle as the bottle drum rotates the receptacles carrying blood culture bottles past the reader station.

41. The method of claim 40, wherein the incubator further comprises indicator LEDs positioned at the distal end of at least a portion of the receptacle in the bottle drum.

42. The method of claim 37, wherein the culture bottles are received in each of the plurality of receptacles in a neck-inward orientation.

43. The method of claim 37, wherein the culture bottles are received in each of the plurality of receptacles in a bottom-inward orientation.
